SEQUENCE LISTING (31 sequences)

SEQ ID NO: 1 - DNA, 104 bp, Artificial Sequence
  Synthetic- SARE
gcacagagccttcctgcgtggggaagctccttgctgcgtcatggctcagctattctcagcctctctccttttatggtgccggaagcaggcaggctgctgctggg

SEQ ID NO: 2 - DNA, 96 bp, Artificial Sequence
  Synthetic- pRAM
ctagaagtttgttcgtgactcagactagaagtttgttcgtgactcagactagaagtttgttcgtgactcagactagaagtttgttcgtgactcaga

SEQ ID NO: 3 - DNA, 420 bp, Artificial Sequence
  Synthetic- ArcMin promoter
gcgcagcagagcacattagtcactcggggctgtgaaggggcgggtccttgagggcacccacgggaggggagcgagtaggcgcggaaggcggggcctgcggcaggagagggcgcgggcgggctctggcgcggagcctgggcgccgccaatgggagccagggctccacgagctgccgcccacgggccccgcgcagcataaatagccgctggtggcggtttcggtgcagagctcaagcgagttctcccgcagccgcagtctctgggcctctctagcttcagcggcgacgagcctgccacactcgctaagctcctccggcaccgcacacctgccactgccgctgcagccgccggctctgctcccttccggcttctgcctcagaggagttcttagcctgttcggagccgcagcaccgacgaccag

SEQ ID NO: 4 - DNA, 103 bp, Artificial Sequence
  Synthetic- cfos promoter
actcattcataaaacgcttgttataaaagcagtggctgcggcgcctcgtactccaaccgcatctgcagcgagcaactgagaagccaagactgagccggcggcc

SEQ ID NO: 5 - DNA, 1428 bp, Artificial Sequence
  Synthetic- Psd95 (delta 1.2)
atggactgtctctgtatagtgacaaccaagaaataccgctaccaagatgaagacacgccccctctggaacacagcccggcccacctccccaaccaggccaattctccccctgtgattgtcaacacggacaccctagaagccccaggatatgagttgcaggtgaatggaacagagggggagatggagtatgagcggatcgtgatccatcggggctccaccggcctgggcttcaacatcgtgggcggcgaggatggtgaaggcatcttcatctccttcatccttgctgggggtccagccgacctcagtggggagctacggaagggggaccagatcctgtcggtcaatggtgttgacctccgcaatgccagtcacgaacaggctgccattgccctgaagaatgcgggtcagacggtcacgatcatcgctcagtataaaccagaagagtatagtcgattcgaggccaagatccatgatcttcgggaacagctcatgaatagtagcctaggctcagggactgcatccttgcgaagcaaccccaagaggggcttctacattagggccctgtttgattacgacaagaccaaggactgcggtttcttgagccaggccctgagcttccgcttcggggatgtgcttcatgtcattgacgctggtgacgaagagtggtggcaagcacggcgggtccactccgacagtgagaccgacgacattggcttcattcccagcaaacggcgggtcgagcgacgagagtggtcaaggttaaaggccaaggactggggctccagctctggatcacagggtcgagaagactcggttctgagctatgagacggtgacccagatggaagtgcactatgctcgtcccatcatcatccttggacccaccaaagaccgtgccaacgatgatcttctctccgagttccccgacaagtttggatcctgtgtccctcatacgacacgtcctaagcgggaatatgagatagacggccgggattaccactttgtctcctcccgggagaaaatggagaaggacatccaggcacacaagttcattgaggctggccagtacaacagccacctctatgggaccagcgtccagtctgtgcgagaggtagcagagcaggggaagcactgcatcctcgatgtctcggccaatgccgtgcggcggctgcaggcggcccacctgcaccccatcgccatcttcatccgtccccgctccctggagaatgtgctagagatcaataagcggatcacagaggagcaagcccggaaagccttcgacagagccacgaagctggagcaggagttcacagagtgcttctcagccatcgtagagggcgacagctttgaagagatctatcacaaagtgaaacgtgtcattgaagacctctcaggcccctacatctgggtcccagcccgagagagactc

SEQ ID NO: 6 - protein, 476 aa, Artificial Sequence
  Synthetic- Psd95 (delta 1.2)
MDCLCIVTTKKYRYQDEDTPPLEHSPAHLPNQANSPPVIVNTDTLEAPGYELQVNGTEGEMEYERIVIHRGSTGLGFNIVGGEDGEGIFISFILAGGPADLSGELRKGDQILSVNGVDLRNASHEQAAIALKNAGQTVTIIAQYKPEEYSRFEAKIHDLREQLMNSSLGSGTASLRSNPKRGFYIRALFDYDKTKDCGFLSQALSFRFGDVLHVIDAGDEEWWQARRVHSDSETDDIGFIPSKRRVERREWSRLKAKDWGSSSGSQGREDSVLSYETVTQMEVHYARPIIILGPTKDRANDDLLSEFPDKFGSCVPHTTRPKREYEIDGRDYHFVSSREKMEKDIQAHKFIEAGQYNSHLYGTSVQSVREVAEQGKHCILDVSANAVRRLQAAHLHPIAIFIRPRSLENVLEINKRITEEQARKAFDRATKLEQEFTECFSAIVEGDSFEEIYHKVKRVIEDLSGPYIWVPARERL

SEQ ID NO: 7 - DNA, 713 bp, Artificial Sequence
  Synthetc- mVenus
gtgagcaagggcgaggagctgttcaccggggtggtgcccatcctggtcgagctggacggcgacgtaaacggccacaagttcagcgtgtccggcgaggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaag

SEQ ID NO: 8 - protein, 236 aa, Artificial Sequence
  Synthetic- mVenus
VSKGEELFTGVVPILVELDGDVNGHKFSVSGEGEGDATYGKLTLKLICTTGKLPVPWPTLVTTLGYGLQCFARYPDHMQHDFFKSAMPEGYVQERTIFFKDDGNYKTRAEVKFEGDTLVNRIELKGIDFKEDGNILGHKLEYNYNSHNVYITADKQKGIKANFKIRHNIEDGGVQLADHYQQNTPIGDGPVLLPDNHYLSYQSKLSKDPNEKRDHMVLLEFVTAAGITLGMDELYK

SEQ ID NO: 9 - DNA, 693 bp, Artificial Sequence
  Synthetic- mScarlet
gtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaag

SEQ ID NO: 10 - protein, 229 aa, Artificial Sequence
  Synthetic- mScarlet
VSKGEAVIKEFMRFKVHMEGSMNGHEFEIEGEGEGRPYEGTQTAKLKVTKGGPLPFSWDILSPQFMYGSRAFIKHPAIPDYYKQSFPEGFKWERVMNFEDGGAVTVTQDTSLEDGTLIYKVKLRGTNFPPDGPVMQKKTMGWEASTERLYPEDGLKGDIKMALRLKDGGRYLADFKTTYKAKKPVQMPGAYNVDRKLDITSHNEDYTVVEQYERSEGRHSTGGMDELYK

SEQ ID NO: 11 - DNA, 1647 bp, Artificial Sequence
  Synthetic- firefly luciferase
gaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtg

SEQ ID NO: 12 - protein, 545 aa, Artificial Sequence
  Synthetic- firefly luciferase
EDAKNIKKGPAPFYPLEDGTAGEQLHKAMKRYALVPGTIAFTDAHIEVDITYAEYFEMSVRLAEAMKRYGLNTNHRIVCSENSLQFFMPVLGALFIGVAVAPANDIYNERELLNSMGISQPTVVFVSKKGLQKILNVQKKLPIIQKIIIMDSKTDYQGQSMYTFVTSHLPPGFNEYDFVPESFDRDKTIALIMNSSGSTGLPKGVALPHRTACVRFSHARDPIFGNQIIPDTAILSVVPHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 13 - DNA, 515 bp, Artificial Sequence
  Synthetic- Nanoluciferase
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgta

SEQ ID NO: 14 - protein, 171 aa, Artificial Sequence
  Synthetic- nanoluciferase
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 15 - DNA, 1014 bp, Artificial Sequence
  Synthetic- TurboID
ggcaagcccatccccaaccccctgctgggcctggacagcaccgctagcaaagacaatactgtgcctctgaagctgatcgctctcctggctaatggcgagttccatagtggcgaacagctgggagaaaccctgggcatgtccagggccgctatcaacaagcacattcagactctgcgcgactggggcgtggacgtgttcaccgtgcccggaaagggctactctctgcccgagcctatcccgctgctgaacgctaaacagattctgggacagctggacggcgggagcgtggcagtcctgcctgtggtcgactccaccaatcagtacctgctggatcgaatcggcgagctgaagagtggggatgcttgcattgcagaatatcagcaggcagggagaggaagcagagggaggaaatggttctctccttttggagctaacctgtacctgagtatgttttggcgcctgaagcggggaccagcagcaatcggcctgggcccggtcatcggaattgtcatggcagaagcgctgcgaaagctgggagcagacaaggtgcgagtcaaatggcccaatgacctgtatctgcaggatagaaagctggcaggcatcctggtggagctggccggaataacaggcgatgctgcacagatcgtcattggcgccgggattaacgtggctatgaggcgcgtggaggaaagcgtggtcaatcagggctggatcacactgcaggaagcagggattaacctggacaggaatactctggccgctacgctgatccgagagctgcgggcagccctggaactgttcgagcaggaaggcctggctccatatctgccacggtgggagaagctggataacttcatcaatagacccgtgaagctgatcattggggacaaagagattttcgggattagccgggggattgataaacagggagccctgctgctggaacaggacggagttatcaaaccctggatgggcggagaaatcagtctgcggtctgccgaaaagctgcagtaa

SEQ ID NO: 16 - protein, 337 aa, Artificial Sequence
  Synthetic- TurboID
GKPIPNPLLGLDSTASKDNTVPLKLIALLANGEFHSGEQLGETLGMSRAAINKHIQTLRDWGVDVFTVPGKGYSLPEPIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAATLIRELRAALELFEQEGLAPYLPRWEKLDNFINRPVKLIIGDKEIFGISRGIDKQGALLLEQDGVIKPWMGGEISLRSAEKLQ

SEQ ID NO: 17 - DNA, 665 bp, Artificial Sequence
  Synthetic- DTE 3′UTR
ttcggctccatgactcagccatgccaggcggagggtcccagaggggctgagtcctcagccccagctgaggcagcagctggagtcttcagagccaggtgaatgacaccaggtctcaagctgctgagaagtctttccggccatgtctggaaggggtaccaccccagcaccagcaccgtcccctcctctcttgaagctgcctgcacagaggttccaagacactttcaaggcagagaaaataggattacaaagaggaggtgccttggcagagggcagcacccagctcagcctcagagctgaaggtgaagacaagccagcgtgaaaccccgggtctgccacgaatgcccgctccgctggccactcaccagctgcctgccacaagccactgcagcttgagcagggtctgtgccctctcagcacagagcccagttcgctgcgtggcctttggcccccgccagaaccttgcaggagccttaaggttcgggccctagcccagcctgaccttacctgctgtgccctgcctgctggtcaagtccagtcccaggagaccccatgccttggctcctaggctgttccaggcacttccctgacctgccgggtgattgcccagctggaacctcatccacaccccagcaccaaccacctcgtgttggtaactgctcgtgtct

SEQ ID NO: 18 - DNA, 122 bp, Artificial Sequence
  Synthetic- SV40 PolyA signal
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 19 - DNA, 241 bp, Artificial Sequence
  Synthetic- tTRE promoter
gagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagaga

SEQ ID NO: 20 - DNA, 747 bp, Artificial Sequence
  Synthetic- d2tTA
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 21 - DNA, 135 bp, Artificial Sequence
  Synthetic- Lck
atgggctgtggctgcagctcaaaccctgaagatgactggatggagaacattgacgtgtgtgagaactgccattatcccggcggacgaattctgcagtcgacggtaccgcgggcccgggatccacgggtcgccacc

SEQ ID NO: 22 - protein, 45 aa, Artificial Sequence
  Synthetic- Lck
MGCGCSSNPEDDWMENIDVCENCHYPGGRILQSTVPRARDPRVAT

SEQ ID NO: 23 - DNA, 37 bp, Artificial Sequence
  Synthetic-MSC
cgcttaattaaggtaccgctagcggcgcgccgaattc

SEQ ID NO: 24 - DNA, 24 bp, Artificial Sequence
  Synthetic- pRAM promoter primer 1
ggcctaactggccgacgcgtaccc

SEQ ID NO: 25 - DNA, 28 bp, Artificial Sequence
  Synthetic- pRAM promoter primer 2
tttaagcttggccgccggctcagtcttg

SEQ ID NO: 26 - DNA, 35 bp, Artificial Sequence
  Synthetic- pRAM promoter amplification primer 1
cgccaccggtgccaccatggactgtctctgtatag

SEQ ID NO: 27 - DNA, 46 bp, Artificial Sequence
  Synthetic- pRAM promoter amplification primer 2
cgtactagtctgggttacctacaaaatcagaacttgtttattgcag

SEQ ID NO: 28 - DNA, 35 bp, Artificial Sequence
  Synthetic- PSD95(delta 1.2) amplification primer 1
ccgcagcaccgacgaccagaagcttgccaccatgg

SEQ ID NO: 29 - DNA, 20 bp, Artificial Sequence
  Synthetic- PSD95(delta 1.2) amplification primer 2
actgcctcgcccttgctcac

SEQ ID NO: 30 - DNA, 20 bp, Artificial Sequence
  Synthetic- mScarlet amplification primer 1
gtgagcaagggcgaggcagt

SEQ ID NO: 31 - DNA, 50 bp, Artificial Sequence
  Synthetic- mScarlet amplification primer 2
cttatcgtcgtcatccttgtagtccttaatcttgtacagctcgtccatgc

(19) United States
(12) Patent Application Publication
(10) Pub. No.: US 2024/0279641 A1
(43) Pub. Date: Aug. 22, 2024

(54) Structural Neuroplasticity Enhancer Screen

(71) Applicant: Northwestern University, Evanston, IL (US)

(72) Inventors: Yevgenia Kozorovitskiy, Evanston, IL (US); Vasin Dumrongprechachan, Evanston, IL (US); FNU Pushpa Kumari, Evanston, IL (US)

(21) Appl. No.: 18/570,782

(22) PCT Filed: Jun. 16, 2022
(86) PCT No.: PCT/US22/33796

§ 371 Date: Dec. 15, 2023

Related U.S. Application Data
(60) Provisional application No. 63/202,581, filed on Jun. 16, 2021.

(51) Int. Cl. C12N 15/10

(52) U.S. Cl. CPC C12N 15/1055

(57) ABSTRACT

The present disclosure describes an activity-dependent regulated polynucleotide capable of detecting dendritic structural plasticity. The present disclosure also describes methods of using in high throughput screens and kits containing the same.

### CROSS-REFERENCE TO RELATED APPLICATIONS

The present application claims priority to U.S. Provisional Patent Application 63/202,581, filed Jun. 16, 2021, the entire contents of which are hereby incorporated by reference.

### STATEMENT REGARDING FEDERALLY SPONSORED RESEARCH

N/A

### REFERENCE TO A SEQUENCE LISTING

This application is being filed electronically via EFS-Web and includes an electronically submitted Sequence Listing in .txt format. The .txt file contains a sequence listing entitled “702581.02162_ST25” created on Jun. 15, 2022 and is 33,950 bytes in size. The Sequence Listing contained in this .txt file is part of the specification and is hereby incorporated by reference herein in its entirety.

### FIELD OF THE INVENTION

The disclosed technology is generally directed to compositions of structural plasticity reporters. More particularly the technology is directed to methods for screening for neuroplasticity.

### BACKGROUND OF THE INVENTION

Our brains contain billions of neurons wired together into complex circuits by trillions of tiny junctions called synapses. Many synapses are located in dendritic spines that stud neuronal dendrites, which serve to collect chemoelectrical signals from other neurons. Dendritic spines are highly specialized membrane protrusions which compartmentalize voltage, ions, and signaling cascades. Dendritic spines vary in density, size, and shape, and they continually undergo morphological and functional changes. Neuronal plasticity-dynamic adjustment in neuronal connectivity—is crucial for brain development, function, and healthy aging. Dysregulated neuroplasticity is central to neurodevelopmental, neurodegenerative, and neuropsychiatric disorders. The search for new compounds that enhance neuroplasticity represents a holy grail for cognitive enhancement and clinical management of human neurological and mental health diseases. The major roadblock today is the absence of efficient means to screen candidate compounds for effects on neuroplasticity. Non-neuronal largescale cellular screens are of limited utility, and phenotypic screening of neurons for changes in dendritic spine shape, size and number is laborious and error prone. Here we solve this problem with a novel high-throughput screening approach yielding a novel discovery and validation pipeline for neuroplasticity modulators.

### BRIEF SUMMARY OF THE INVENTION

The present disclosure describes an activity-dependent polynucleotide capable of detecting dendritic structural plasticity.

In one aspect, the disclosure provides an activity-dependent regulated polynucleotide capable of detecting dendritic potentiation comprising one or more of the following: (a) an activity-response element, (b) an activity-dependent promoter, (c) a subcellular targeting motif, (d) one or more reporters, and (e) an mRNA targeting element. In some aspects, the polynucleotide comprises two or more of (a)-(e), alternatively three or more of (a)-(e), alternatively four or more of (a)-(e), alternatively comprising (a)-(e) (all five components).

In another aspect, the disclosure provides an activity-dependent regulated polynucleotide capable of detecting dendritic potentiation comprising one or more of the following: (a) an activity-response element, (b) an activity-dependent promoter, (c) a subcellular targeting motif, (d) a fluorescent reporter or biotinylating reporter, (e) an enzyme reporter, and (f) an mRNA targeting element. In some aspects, the polynucleotide comprises two or more of (a)-(f), alternatively three or more of (a)-(f), alternatively four or more of (a)-(f), alternatively five or more of (a)-(f), alternatively comprising (a)-(f) (all six components).

In another aspect, the disclosure provides a construct capable of introducing the polynucleotide described herein into a host cell, the construct comprising the polynucleotide described herein.

In another aspect, the disclosure provides a virus vector comprising the polynucleotide of or construct described herein.

In a further aspect, the disclosure provides a host cell expressing the polynucleotide or construct described herein.

In another aspect, the disclosure provides a high throughput method of testing a therapeutic molecule capable of altering dendritic potentiation, the method comprising: (a) contacting a cell expressing the polynucleotide or construct described herein with a therapeutic molecule; and (b) detecting the reporter expression in the cell, wherein the expression of the reporter is indicative of dendritic or synaptic potentiation.

In another aspect, the disclosure provides a kit comprising as components: (a) the polynucleotide or construct described herein; and (b) instructions for use in detecting the reporter expression in a cell, wherein the expression of the reporter is indicative of dendritic or synaptic potentiation of the cell.

In another aspect, the polynucleotides comprise: (a) an activity-response element (for example, SEQ ID NO: 1-2) (b) an activity-dependent promoter (for example, SEQ ID NO: 3-4), (c) a subcellular targeting motif (for example, SEQ ID NO: 5-6), (d) a fluorescent reporter (for example, SEQ ID NO: 7-10), (e) an enzyme reporter (for example, SEQ ID NO: 11-16), (f) and an mRNA targeting element (for example, SEQ ID NO: 17). The construct is designed to selectively target dendritic spines and report activity-induced plasticity as luminescence. The polynucleotide construct is flexible and can be modified, but these components help detect dendritic structural plasticity in response to neural activity in a high-throughput manner. Some components may be omitted or modified to retain construct functionality. Suitably, in one example, the activity-response element is a synaptic activity response element, the activity-dependent promoter may be Arc, the subcellular targeting motif may be Psd95(Δ1.2) (e.g., Seq ID NO: 5), the fluorescent reporter may be mScarlet, the enzyme reporter may be luciferase and a dendritic targeting element.

The present disclosure further describes methods of using the polynucleotide including a high throughput method within a multi well plate to screen for pharmaceutical molecules that can activate dendritic structural plasticity and a kit containing the same.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1A-1C. Concepts and map of structural plasticity reporter. A) General design of structural plasticity reporters. A structural plasticity reporter consists of five components including an activity-dependent promoter, a subcellular targeting motif, a fluorescent reporter, an enzyme reporter, and mRNA targeting element. B) Map of SARE-Arc-Luc construct. C) Basic anatomy of a dendrite

FIG. 2A-2C. Designs and testing of structural plasticity-dependent constructs. A) Design of firefly luciferase-based structural plasticity-dependent constructs (Arc, cFos promoters for two major activity-dependent signaling pathways. d2-tTA::TRE reporter amplification cassette (SEQ ID NO: 19-20). B) Expression of firefly luciferase-based structural plasticity-dependent constructs in HT22 cells. Epifluorescence images of HT22 cells expressing all constructs via transfection. Scale bar: 40 μm. C) Dose-response relationship in response to forskolin stimulation for each construct in (a). n=3, mean±SEM. Curve was fitted with [agonist] vs. response with Hill slope=1, and plotted with log 10-transformed [agonist].

FIG. 3A-3C. Targeting structural plasticity compartments in mammalian neural circuits. A) Schematic of high-throughput assay protocol in primary culture neurons. B) Expression of luciferase-PSD95-mVenus in live DIV14 neurons (scale-10 μm). C) Dose-response relationship in response to known plasticity-promoting agents for SARE-ArcMin-luciferase-PSD95-mVenus. n=3, mean±SEM. Curve was fitted with [agonist] vs. response with Hill slope=1, and plotted with log 10-transformed [agonist].

FIG. 4A. Structural plasticity modulation validation in cortical primary neuronal culture. A) Dose-response relationship in response to known plasticity-promoting agents (Forskolin and Gabazine) and their inhibitors for SARE-ArcMin-luciferase-PSD95-mVenus. Forskolin and Gabazine was added with their corresponding pathway inhibitors (H89 and TTX respectively) for 16 hrs before luciferase assay. n=3, mean±SEM. Curve was fitted with [agonist] vs. response with Hill slope=1, and plotted with log 10-transformed [agonist].

FIG. 5A-5C. Validation of NanoLuciferase-mScarlet response in HT22 hippocampal cell line culture. A) AS.mScarlet. NanoLuc expression in HT22 cells. Epifluorescence images of HT22 cells expressing pAAV-SARE. ArcMin.AS.mScarlet. NanoLuc via transfection. Scale bar: 40 μm. B) Western blot analysis of HT22 cell lysates. NanoLuc construct detected by anti-RFP antibody was observed at expected size (˜100 kDa). Qualitatively, expected increase in NanoLuc expression after Forskolin (FSK) treatment cannot be distinguished from vehicle control by western blot. C) Dose-response curve for NanoLuc responses to forskolin stimulation. n=3, mean±SEM. Curve was fitted with [agonist] v.s. response with Hill slope=1, and plotted with log 10-transformed [agonist]. Assays were repeated twice, independently. Overall, luciferase readout offers greater sensitivity and reproducibility to detect increase in NanoLuc expression compared to western blot analysis.

FIG. 6A-6E. High-throughput assay setup for screening plasticity modulating agents. A) Design of structural plasticity-dependent reporter for high throughput screening. Synaptic activity-responsive element (SARE) (SEQ ID NO: 1) and proximal Arc promoter (SEQ ID NO: 3) along with a 3′-dendritic targeting element (DTE) (SEQ ID NO: 17) was used for activity-dependent expression. Psd95 with PDZ 2-3 (SEQ ID NO: 5-6) domains deleted was fused to mScarlet-NanoLuc for localization to dendrites and dendritic spines. The construct was packaged into AAV1. High throughput assay was set up in 384 wells plated with primary mixed cortical neuron cultures. Neurons were transduced at DIV 3 with AAV1.AS.mScarlet.NanoLuc (dose: MOI 5). Pharmacological agents were added at DIV 21, followed by luciferase readout 16 hr post treatment. B) Activity-dependent AS.mScarlet.NanoLuc expression. Confocal images show increased AS.mScarlet. NanoLuc expression at DIV 14, 16 hr. after 1 μM forskolin treatment compared to 0.1% DMSO vehicle control. Scale bar: 20 μm. C) Localization of AS.mScarlet.NanoLuc in dendrites. Activity-dependent AS.mScarlet.NanoLuc localizes to a subset of dendritic spines. Scale bar: 5 μm. D) Quantitative luciferase reporter readout of AS.mScarlet. NanoLuc expression. Dose-response relationship of AS.mScarlet.NanoLuc in response to forskolin stimulation at DIV 21, 16 hr post stimulation. n=3, mean±SEM. Curve was fitted with [agonist] v.s. response with Hill slope=1, and plotted with log 10-transformed [agonist]. E) Western blot analysis of forskolin stimulation in cell culture. Increased cFos expression was observed after FSK treatment (FSK−DMSO=3.232±0.769, n=3, adj. p=0.0462). An apparent increase in NanoLuc expression was observed (FSK−DMSO=0.583±0.267, n=3, adj. p=0.324) but more replicates are needed for western blot analysis compared to luciferase readout. No difference was observed for Psd95 (FSK−DMSO=−0.048±0.1131, n=3, adj. p=1). Unpaired t-test with Bonferroni correction.

FIG. 7A-7B. Viral transduction dose optimization for 384-well plate format and stimulation validation. A) Viral transduction dose optimization for 384-well plate format. Neurons were transduced with CAG.GFP or AS.mScarlet. NanoLuc at MOI=1, 2, 5. At DIV21, cells were stimulated with 0.5, 25 μM forskolin (FSK) or 0.002%, 0.1% DMSO vehicle control. Activity-dependent responses were only observed for the NanoLuc construct at MOI=2 and 5. NanoLuc at MOI 5 shows the most robust response and was used in subsequent experiments. B) Summary data for FIG. 6e. n=3, mean±SEM. Unpaired t-test with Bonferroni correction. Adjusted p-values=0.3237, 0.0462, and 1 for RFP, cFos, and Psd95, respectively.

$$MOI = \frac{Virus Titer ( Genome copy ) \times Volume of virus}{Total Cell Number}$$

FIG. 8A-8C. High-throughput assay uniformity and reproducibility based on NIH guidelines. A) Plate layout in an interleaved-signal format to produce a combination of “Max”, “Min” and “Mid” signals. Two different concentrations of Forskolin (For Max and Mid signals) and a DMSO control (0.1%) were used for statistical analysis. B) Average Luminescence of all conditions used (n=96 data points per condition). C) Z′-factor calculation; Negative control (0.1% DMSO) and positive controls (2.5 and 0.5 μM forskolin, FSK) were interleaved by columns in a 384-well plate (n=96 wells per condition). Reporter activity was measured and plotted in order of wells from left to right. Drift and edge effects were not significant across the plate. Robust z′-factors of both mid and high doses of FSK response compared to DMSO indicate sufficient separation between positive and negative controls. Z′-factor of the assay is well above the standard acceptance criteria of z′-factor>0.4.

$${Z}^{′} = \frac{( {AVG}_{max} - 3 {SD}_{max} / √ n ) - ( {AVG}_{min} + 3 {SD}_{min} / √ n )}{{AVG}_{max} - {AVG}_{min}}$$

FIG. 9A-9B. Expression of Nanoluciferase-mScarlet in vivo in the adult mouse brain. A) Schematic of unilateral stereotactic viral transduction in adult Thy 1-GFP mice. Mice were injected 500 nl of AAV2/1-SARE-ArcMin-PSD95-Nanoluciferase-mScarlet (1.5×10¹³ GC) in hippocampus unilaterally. Non-injected site was considered as no AAV control. Brain samples were harvested 21 days after injection (n=2). B) Expression of Nanoluc-mScarlet in a coronal brain section. Scale bar: 200 μm.

FIG. 10A-10C. Validation of TurboID based plasticity sensors in HT22 cells A) Proximity labeling constructs for mapping potentiated dendritic proteome: (1) activity-dependent, (2) constitutive control. B, C) biotinylation activity in HT22 cells. (b) biotin-labelled protein detection in a western blot (c) biotinylation in fixed cells.

FIG. 11A-11C. TurboID based dendritic plasticity reporters in primary cortical neurons. A) Design for proteomic reporter for dendritic plasticity. Compartment specific structural plasticity constructs for proximity labelling of proteome (1) SARE-Arc-TurboID—Activity-dependent spine targeting construct; (2) LckTurboID—constitutive membrane control (SEQ ID NO: 21-22) B) Biotin labeling in compartment specific manner for constructs (1)-(2) in primary cortical neurons (overlayed with AAV-CAG-EGFP expression). Sparse, punctate biotin labeling in construct SARE-Arc-TurboID. (1) shows selective labeling of proteins in dendritic spines, while Lck-TurboID (2) is targeted to plasma membrane bound proteome. Scale bar: 20 μm. C) Western blot analysis of biotinylation pattern of constructs in cortical neurons. The results suggested enhanced biotin-labelling after induction of neuronal activity by 55 mM KCl. Induction of activity was validated by change in expression of PSD95.

FIG. 12A-12B. Validation of dendritic targeting and labelling of synaptic proteins. A) Sample preparation workflow of biotin labelling and sub-cellular fractionation. B) Western blot analysis of fractions of synaptosomal preparation-S1, (total homogenate), P1 (nuclear-protein enriched), S2 (Cytosolic-proteins enriched), and P2 (Synaptic-proteins enriched) fractions. Increased biotinylation was detected in P2 fraction in comparison with S2 and P1 fraction indicates labelling of synaptic proteins in SARE-Arc-TurboID. Alpha tubulin was used for protein loading control (1:1000). Enhanced expression of Synaptophysin and depleted expression of Histone H3 suggested synaptosomal fraction was enriched in synaptic proteins and devoid of nuclear proteins.

### DETAILED DESCRIPTION OF THE INVENTION

Despite the extensive body of research focused on synaptic mechanisms in mammalian neurons, there are no screening tools capable of performing dynamic measurements of neuroplasticity in a high-throughput format. The present disclosure provides a novel plasticity biosensor (i.e., activity-dependent plasticity reporters) and a high-throughput screening pipeline for discovering neuroplasticity modulators and mechanisms.

The present disclosure provides a genetically encoded biosensor design (e.g., polynucleotide), where the translation of a high-throughput reporter, e.g., luciferase, has activity-dependent regulation to produce a signal correlated with normal synaptic activation. The polynucleotide sensor is targeted to neuronal synapses for expression of the reporter. The biosensor design of the present disclosure comprises an activity-dependent polynucleotide capable of detecting dendritic structural plasticity. Thus the biosensor and cells comprising the biosensor can be used as a screening tool to look for molecules that may alter dendritic structural plasticity.

In one embodiment, the polynucleotide comprises one or more of the following: an activity-response element, an activity-dependent promoter, a subcellular targeting motif, a fluorescent reporter, an enzyme reporter and an mRNA targeting element, preferably two or more of the components, alternatively three or more of the components, alternatively four our more of the components, alternatively five or more components, alternatively all 6 components. Thus the polynucleotide is transcribed and translated in a cell when a molecule activates the dendritic spine, demonstrating dendritic structural plasticity and dendritic potentiation. Neurons are polarized cells that exhibit a high degree of spatial compartmentalization. Dendrites of many neuronal classes are studded with small protrusions called dendritic spines, which compartmentalize postsynaptic machinery, and the magnitude of excitatory input received by a neuron depends on the complexity of dendritic arbors, density, and size of dendritic spines. Thus, neuroplasticity at the level of dendritic spines is fundamental to the development and function of neural circuits and thus detection can be used as a biosensor for neuroplasticity and dendritic potentiation. A dendritic spine is a small, club-like cell protrusion from neuronal dendrites that form the postsynaptic component of most excitatory synapses in the brain. Spines are complex, dynamic structures that contain a dense array of cytoskeletal, transmembrane, and scaffolding molecules. Dendritic spines typically receive input from a single axon at the synapse and serve as a storage site for synaptic strength and help transmit electrical signals to the neuron's cell body. As used herein, dendritic potentiation may also be described as dendritic structural plasticity. These terms refer to the dynamic nature of the dendritic spines and ability of neural networks in the brain to change through growth and reorganization. Deficits in dendritic spine plasticity are central to neurodevelopmental, neurodegenerative, and neuropsychiatric disorders and the biosensor, construct and cells described herein can be used for high-throughput screening of compounds that may provide therapeutic benefits in neurodegenerative or neuropsychiatric disorders related to neuroplasticity.

In one embodiment, the disclosure provides an activity-dependent regulated polynucleotide capable of detecting dendritic potentiation comprising one or more of the following: (a) an activity-response element, (b) an activity-dependent promoter, (c) a subcellular targeting motif, (d) one or more reporters, and (e) an mRNA targeting element. In some aspects, the polynucleotide comprises two or more of (a)-(e), alternatively three or more of (a)-(e), alternatively four or more of (a)-(e), alternatively comprising (a)-(e) (all five components).

In another embodiment, the disclosure provides an activity-dependent regulated polynucleotide capable of detecting dendritic potentiation comprising one or more of the following: (a) an activity-response element, (b) an activity-dependent promoter, (c) a subcellular targeting motif, (d) a fluorescent reporter or biotinylating reporter, (e) an enzyme reporter, and (f) an mRNA targeting element. In some aspects, the polynucleotide comprises two or more of (a)-(f), alternatively three or more of (a)-(f), alternatively four or more of (a)-(f), alternatively five or more of (a)-(f), alternatively comprising (a)-(f) (all six components).

The present disclosure describes a polynucleotide encoding an activity-dependent plasticity reporter system. A polynucleotide is a biopolymer comprised of a long, linear series of nucleotides joined together by ester linkages between the phosphoryl group of nucleotides and the hydroxyl group of the sugar component of the next nucleotide.

The polynucleotide described herein, may comprise an activity-response element. As used herein, an activity-response element are unique genomic structures that respond with high sensitivity to cellular activity. These activity response elements may be a synaptic activity response element (SARE). SARE possesses a strong enhancer activity that is uniquely sensitive in response to synaptic stimulation. SARE locates in an evolutionarily conserved genomic region in the Arc promoter. A robust activity marking (RAM) system may also be used. RAM allows for the identification and interrogation of ensembles of neurons.

The reporter system comprising the polynucleotide described herein, may comprise an activity dependent promoter. As used herein, an activity-dependent promoter controls the expression of neuronal immediate early genes which are rapidly induced neuronal activity. Activity dependent promoters used herein may include Arc, cFos, Npas4, Egr1, cJun or other immediate early gene promoters. Suitable promoters would be understood by one skilled in the art. The polynucleotide described herein, may comprise a targeting motif. The targeting motif may be a subcellular targeting motif. Targeting motifs transport proteins to their appropriate destination, for example, to the dendrites. A targeting motif used herein includes Psd95(Δ1.2)(SEQ ID NO 5-6). Other targeting motifs include homer 1 or bassoon (bsn), or other known targeting motifs in the art.

The polynucleotide described herein, may comprise a reporter. A reporter is often in contact with a regulatory sequence or gene and confers characteristics that are easily identified and measured. These characteristics may be an indicator of activity or the state of the regulatory sequence the reporter is in contact with. The reporter may be a fluorescent reporter, a luminescent reporter, or a biotinylating reporter.

The polynucleotide described herein, may comprise a fluorescent reporter. A fluorescent reporter codes for a protein that has a characteristic fluorescence emission spectrum when excited with light at a specific wavelength. As used herein the fluorescent reporter may include mVenus (for example, but not limited to, SEQ ID NO: 7-8), mScarlet (for example, but not limited to, SEQ ID NO: 9-10). The florescent reporter may be a fluorescent acceptor.

The polynucleotide described herein, may comprise an enzyme reporter. Enzyme reporters code for proteins that have a unique enzymatic activity and are used to assess the transcriptional properties of DNA elements. Enzyme reporters used herein include firefly luciferase, Nano Luciferase, synthetic (dCpG) Luciferase, Click-beetle red luciferase, alkaline phosphatase, horse radish, and ascorbate peroxidases, or similar enzymes. For example, as demonstrated in the examples, enzyme reporters can be firefly luciferase (e.g., SEQ ID NO: 11-12), Nano Luciferase (e.g., SEQ ID NO: 13-14). The enzyme reporter may be a bioluminescent donor.

The polynucleotide described herein, may comprise an mRNA targeting element. The mRNA may be selectively targeted to dendrites. The mRNA targeting element may be a dendritic targeting element. The dendritic targeting element may comprise DTE-SV40. For example, but not limited to, DTE-SV40 comprises SEQ ID NO: 17-18 or a sequence having at least 90% homology to SEQ ID NO: 17-18.

The polynucleotide described herein, may comprise a biotin ligase. In some embodiments, the enzyme reporter is replaced by a biotin ligase. In some embodiments, the biotin ligase is and engineered biotin ligase including TurboID (for example, SEQ ID NO: 15-16) BioID, BioID2, AirID, or miniTurbo.

Some embodiment describes a construct capable of introducing the polynucleotide into a host cell, the construct comprising the polynucleotide described herein. Suitably, the construct may be a vector.

The fusion proteins produced by the methods described herein are also contemplated.

In some embodiments, vectors are provided. A vector is any particle used as a vehicle to artificially carry a foreign nucleic sequence, typically DNA into another cell, where it can be replicated and/or expressed. A vector containing foreign DNA is termed recombinant DNA. The four major types of vectors are plasmids, viral vectors, cosmids, and artificial chromosomes. The vector may be for example a viral vector. Suitable viral vectors are known in the art and include, for example, an Adeno-Associated viral (AAV) vector. A vector may contain a promoter, operably linked to any one of the polynucleotides described herein. As used herein, a polynucleotide is “operably connected” or “operably linked” when it is placed into a functional relationship with a second polynucleotide sequence.

In some embodiments, a host may comprise the polynucleotide or AAV-vector associated polynucleotide described herein. In particular, the host cell may be a neuronal cell or neuron. A polynucleotide or a vector containing a reporter system comprising the polynucleotide may be administered to the host, e.g., host cell. Administration of the vector or polynucleotide to the host cell can be carried out using various mechanisms known in the art, including naked administration and administration in pharmaceutically acceptable carriers. Polynucleotides may be administered to host cell via transfection, biolistic gene delivery, microneedle system of injecting, electroporation, gene gun or magnetic-assisted transfection. Vectors may be administered to the host via transduction, including lentiviral transduction.

The present disclosure further describes a high throughput method of testing a therapeutic molecule capable of altering dendritic potentiation (e.g., synaptic plasticity). This method allows for the quick screening for active compounds in a particular biomolecular pathway, such as dendritic plasticity. In some embodiments the therapeutic molecule is any neuroactive substance. Suitable, these may include any molecule that is thought or suggested to alter dendritic plasticity. These therapeutics may include forskolin, ketamine, gabazine, psilocybin, serotonergic, psychedelic, entactogen compounds, psychostimulants, antipsychotics or other psychoactive compounds.

The high throughput screen described herein can be used to screen pharmacological agents for neuroplasticity capabilities to address neurodevelopmental diseases, mental health diseases, cognitive enhancement strategies, and personalized medicine neuroplasticity applications. Further, the high throughput screen described herein can be used to screen pharmaceutical agents in vitro using cell lines of neuronal origin (e.g. HT22 cell line), primary neuronal cells, neural stem cells, neural organoids, patient derived pluripotent stem cells (iPSCs) differentiated into neurons or neural organoids, and in vivo animal models primarily rodents including genetically modified transgenic mice.

Further, the biosensor and assay described herein can directly screen large scale drug libraries for their potential to enhance neuroplasticity with a biochemical, rather than imaging, readout. Diverse human neurological and mental health problems are linked to disturbances in synaptic plasticity. While some specific pharmacological compounds are known to enhance plasticity, there are no high throughput ways to screen compounds for neuroplasticity before the present disclosure.

In some embodiments multiple therapeutic agents may be contacted to the polynucleotide expressing cell in a multi-well plate. The plate may be flat with multiple wells. The plate may contain 4, 6, 12, 24, 48, 96, 384 or 1536 wells. Interactions between a polynucleotide described herein and a therapeutic agent take place in each well. The therapeutics may be administered once or more times, and at multiple concentrations.

Another aspect of the present disclosure provides a kit comprised of a polynucleotide described herein capable of detecting dendritic structural plasticity. In some embodiments the kit comprises a multi-well plate comprising neuronal cells expressing the polynucleotide reporter system. The cells can be contacted with a therapeutic molecule to be tested, and the dendritic plasticity can be measured via the reporter system and detection of the enzymatic reporter (e.g., luciferase). The kit can include instructions for use.

In some embodiments, the kit includes a packaging material. As used herein, the term “packaging material” can refer to a physical structure housing the components of the kit. In some instances, the packaging material maintains sterility of the kit components, and is made of material commonly used for such purposes (e.g., paper, corrugated fiber, glass, plastic, foil, ampules, etc.). Other materials useful in the performance of the assays are included in the kits, including test tubes, transfer pipettes, and the like. In some cases, the kits also include written instructions for the use of one or more of these reagents in any of the assays described herein.

In some embodiments, kits also include a buffering agent, a preservative, or a protein/nucleic acid stabilizing agent. In some cases, kits also include other components of a reaction mixture as described herein. In some cases, kits also include a control sample and/or includes a negative control sample and/or a positive control sample.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user. The term “substantial identity” of polynucleotide and amino acid sequences for purposes of this invention normally means sequence identity of at least 40%. Preferred percent identity of polynucleotides or polypeptides can be any integer from 40% to 100%. More preferred embodiments include at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100%.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example I—Structural Neuroplasticity Enhancer Screen

Neurons are polarized cells that exhibit a high degree of spatial compartmentalization. During development, they undergo elaborate changes in dendritic arborization and connectivity. Dendrites of many neuronal classes are studded with small protrusions called dendritic spines, which compartmentalize postsynaptic machinery. The magnitude of excitatory input received by a neuron depends on the complexity of dendritic arbors, density, and size of dendritic spines^{1, 2}. Thus, neuroplasticity at the level of dendritic spines is fundamental to the development and function of neural circuits. Structural and proteomic changes of dendritic spines are broadly considered the basis of learning and memory³, where larger spines correlate with greater synaptic strength⁴. In the developing brain, the turnover rate of spines is fast, facilitating neural circuit formation and refinement. In the adult, a large fraction of dendritic spines is mature and more stable⁵. Deficits in dendritic spine plasticity are central to neurodevelopmental, neurodegenerative, and neuropsychiatric disorders^{6,7}. Aberrant dendritic spine density in multiple cortical regions during development is associated with intellectual disability and Autism Spectrum Disorders⁸, while progressive alterations in dendritic spine morphology and a global loss of spines and synaptic markers are correlates of cognitive impairment⁹. Since disruption of dendritic spine and synapse regulation underlies disease pathology, neuroactive agents that modulate structural plasticity are relevant as therapeutics and cognitive enhancers. Recent studies indicate the involvement of pharmacological agents, like ketamine, psilocybin, serotonergic, psychedelic, and entactogen compounds, in altering the structural plasticity of dendritic spines and excitatory synaptic transmission, useful for treatment of neuropsychiatric disorders^{10,11,12}. Structural plasticity modulators encompass diverse classes of compounds¹³. Despite the extensive body of research focused on synaptic mechanisms in mammalian neurons, there are no screening tools capable of performing dynamic measurements of neuroplasticity in a high-throughput format. Here we solve this problem with a novel plasticity biosensor and a high-throughput screening pipeline for discovering neuroplasticity modulators and mechanisms.

This invention is based on a genetically encoded biosensor design, where the translation of a high-throughput compatible reporter such as luciferase enzyme is subjected to the same control as normal synaptic activation. The sensor is targeted neuronal synapses for expression. We demonstrate high-throughput readouts that are compatible with various models of neuronal cells including primary neuronal cultures from rodent brains, patient-derived pluripotent stem cells differentiated into neurons, brain organoids, and animal brains.

### Technical Description

To develop a high throughput assay to measure dendritic potentiation in primary neuronal cultures, the assay must meet two criteria: high throughput compatibility and a readout that indicates dendritic potentiation. To achieve this, we turned to a genetically encoded biosensor design where the translation of a high-throughput reporter, luciferase, is subjected to the same activity-dependent regulation as normal synaptic activation. First, we fused luciferase (e.g., firefly luciferase (e.g., SEQ ID NO: 11-12), or Nano Luciferase (e.g., SEQ ID NO: 13-14) to a synaptically localized fluorescent tag Psd95(Δ1.2) (e.g., SEQ ID NO: 5-6)-mVenus ((e.g., SEQ ID NO: 7-8) under the control of an activity-dependent promoter (e.g., transcription factors Arc, cFos (e.g., SEQ ID NO: 3-4)). The 5′-end after the stop codon contains a dendritic targeting element (DTE) (e.g., SEQ ID NO: 17-18) mRNA sequence. Due to its dendritic and spine localization, the regulation of Psd95-luciferase reporter—translation, localization, and degradation—should be similar to endogenous Psd95. Deletion of PDZ domains 1 and 2 (Δ1.2) ensures that reporter expression does not interfere with normal synaptic activity. We initially created three structural plasticity-dependent constructs as a proof of concept and tested them in the hippocampus-derived, immortalized HT22 cell line by chemical transfection with lipofectamine or polyethylenimine-based methods. Forskolin (FSK), a known positive plasticity modulator, was added to media at different concentrations. The Arc pathway construct (1) shows the strongest response to FSK stimulation. Construct (3), with an amplification cassette based on construct (2), has a dose-response behavior similar to (2), but with a greater signal. A combination of promoters (e.g., Arc, cFos, Npas4, Egr1), synaptic proteins, fluorescent proteins, and luciferases can improve reporter localization selectivity, and signal for high throughput screens beyond these constructs. Cell lines have limited utility, especially for neuroplasticity investigation. Because immortalized cell lines do not produce action potential or form stable synapses, they are not a good model to conduct neuroplasticity screening. Instead, we optimize the assay for primary neuronal culture. We followed the NIH assay guidance manual to ensure maximal quality and reproducibility. For optimal expression of the plasticity reporter construct (1), several modifications were made to improve adeno-associated virus (AAV) packaging ability, to increase luciferase readout sensitivity for small number of cells (e.g., 1000 cells per well in 384 well plates), and to include a bright photostable fluorescent proteins. We created pAAV-SARE-ArcMin-PSD95(Δ1.2)-mScarlet-NanoLuc-DTE (AS.mScarlet.NanoLuc for short). mScarlet (SEQ ID NO: 9-10) is a bright monomeric fluorophore, chosen over mVenus because of its non-overlapping spectral properties with EGFP and Alexa647, facilitating microscopy experiments. NanoLuc (SEQ ID NO: 13-14) and its substrate also provide an extremely sensitive and stable signal in a 384-well plate set up, where only limited number of cells can be plated. Therefore, this new construct can be packaged into AAV for reproducible expression of the plasticity biosensor in cell culture, patient-derived inducible pluripotent stem cells, brain organoids, and animal brains. After cell plating and AAV dose optimization, we demonstrated dendritic and spine localization of Psd95(Δ1.2)-mScarlet-NanoLuc, as well as a dose-response relationship with a positive plasticity modulator FSK. We carried out a quality control experiment to determine the z′-factor using FSK and 0.1% DMSO as positive and negative controls. Altogether, we have created a series of plasticity-dependent reporters to measure dendritic potentiation and demonstrated its high-throughput application for screening molecules. In addition to using primary neuronal culture, these methods can be readily used for patient-derived inducible pluripotent stem cells, brain organoids, and animals in vivo, while the plasticity reporter can be introduced into cells by chemical transfection, electroporation, viral vector transduction, or gene editing. A combination of promoters, synaptic proteins, fluorescent proteins, and luciferases can be combined with transgenic mouse-derived neurons for cell type-specific readout, and relevance to disease models. This allows screening for neuroplasticity for neurons harboring specific mutations for personalized medicine applications.

### Methods

### Plasmid Construction

pAAV-SARE.ArcMin-PSD95(Δ1.2)-mVenus-MCS-DTE was synthesized by Genscript based on Hayashi-Takagi et al. Briefly, SARE.ArcMin promoter (SEQ ID NO: 1 and 3) was based on Kawashima et al. (104 bp synaptic activity-responsive element, −6793 to −6690, and 421 bp, −222 bp to +198 bp, of mouse Arc Arg-3.1 gene (Kawashima et al., 2009). PSD-95(ΔPDZ1.2) (SEQ ID NO: 5-6) was generated by deleting the nucleotides (nts) 250 to 993 based on the numbering of NM_019621. DTE sequence (SEQ ID NO: 17) was from 2036 bp to 2699 bp from NM_019361. MCS sequence ((CGCTTAATTAAGGTACCGCTAGCGGCGCGCCGAATTC) (SEQ ID NO: 23) was inserted between mVenus (SEQ ID NO: 7-8) and DTE. Firefly luciferase sequence (SEQ ID NO: 11-12) (a gift from Dr. Geoff Wahl, Addgene #11685) was inserted between PacI and NheI, in frame with mVenus to generate pAAV-SARE.ArcMin-PSD95(Δ1.2)-mVenus-ffLuc-DTE. For pRAM (SEQ ID NO: 2)-containing constructs, pRAM promoter was amplified from pAAV-RAM-d2TTA::TRE-FLEX-tdTomato-WPREPA (a gift from Dr. Yingxi Lin, Addgene #84468) and replace SARE.ArcMin promoter between MluI and HindIII using 5′-ggcctaactggccgacgcgtaccc-3′ (SEQ ID NO: 24) and 5′-TTTAAGCTTggccgccggctcagtcttg-3′ (SEQ ID NO: 25) to generate pAAV-pRAM-PSD95(Δ1.2)-mVenus-ffLuc-DTE. To create an amplification design, pAAV-RAM-d2TTA::TRE-FLEX-PSD95(Δ1.2)-mVenus-ffLuc-WPREPA, FLEX-tdTomato-WPRE was replaced by PSD95(Δ1.2)-mVenus-ffLuc between AgeI and SpeI sites using 5′-cgccaccggtgccaccatggactgtctctgtatag-3′ (SEQ ID NO: 26) and 5′-cgtactagtctgggttacctacaaaatcagaacttgtttattgcag-3′ (SEQ ID NO: 27). pAAV-SARE.ArcMin-PSD95(Δ1.2)-mScarlet-ffLuc-DTE was generated by Gibson assembly. The first fragment (PSD95(Δ1.2)) was amplified using 5′-ccgcagcaccgacgaccagAAGCTTgccaccatgg-3′ (SEQ ID NO: 28) and 5′-ACTGCCTCGCCCTTGCTCAC-3′ (SEQ ID NO: 29). The second fragment (mScarlet) was amplified from pCAG-FLEX-mScarlet-WPRE (a gift from Dr. Ryan Larsen, Addgene #99280) using 5′-GTGAGCAAGGGCGAGGCAGT-3′ (SEQ ID NO: 30) and 5′-CTTATCGTCGTCATCCTTGTAGT CCTTAATCTTGTACAGCTCGTCCATGC-3′ (SEQ ID NO: 31). Two fragments replace PSD95(Δ1.2)-mVenus in pAAV-SARE.ArcMin-PSD95(Δ1.2)-mVenus-ffLuc-DTE between HindIII and PacI. Lastly, NanoLuc gene block was synthesized by Integrated DNA technologies, and inserted into pAAV-SARE.ArcMin-PSD95(Δ1.2)-mScarlet-ffLuc-DTE between BsrGI and NheI sites to create the final construct pAAV-SARE.ArcMin-PSD95(Δ1.2)-mScarlet-NanoLuc-DTE which is optimal for AAV packaging, imaging, and luciferase assay.

### HT22 Mouse Hippocampal Cell Cultures

HT22 cells were obtained from the Salk Institute and cryorecovered in complete Dulbecco's Modified Eagle Medium (DMEM) supplemented with 10% fetal bovine serum (FBS) and 100 U/mL penicillin-streptomycin in 37° C./5% CO₂ incubator (Cat. No. 11965118, 10437028, 15140122, Thermo Fisher). Transfection was performed using linear 25 k polyethylenimine (Cat. No. 23966-1, Polysciences, Warrington, PA) for luciferase assay in 96 well plates and for western blot in 6 well plates. PEI to DNA ratio was 7 μg PEI to 1 μg DNA (200 ng of DNA for each well in a 96 well plate and 3 μg DNA for each 10 cm dish). PEI was added to DNA prediluted in OptiMEM (Thermo Fisher, Cat. No. 31985062) followed by vigorous vortexing. DNA-PEI complex was incubated at RT for 15 min and added drop-wise to cells. On the following day, media was completely changed to OptiMEM with or without any ligand for end point experiments. Ligand concentration or vehicle control was specified in figure legends. Cells were imaged with Biotek Lionheart LX automated microscope system.

### Primary Mixed Cortical Cultures

Animals were handled according to protocols approved by the Northwestern University Animal Care and Use Committee. Cortical neurons were harvested from mouse embryos euthanized at embryonic day 18 and plated in poly-D-lysine coated plates. Embryonic brains were dissected in ice-cold dissection media (Hank's balance salt solution HBSS, 2% penicillin-streptomycin, 20 mM HEPES). Meninges were removed from each brain and cortices were dissected out. The cortices were chopped into small pieces using a microscissors and pooled in 5 mL dissection media containing 0.25% trypsin and 0.1% DNasel and trypsinised at 37° ° C. for 20-25 min. Cortices were washed twice with warm dissection media, and once with warm plating medium (MEM, supplemented with 10% heat-inactivated horse serum, 0.6% glucose, 1 mM Na pyruvate, 1% Glutamax, 1% penicillin-streptomycin) for 5 min each. Tissues were titurated in 3 mL fresh plating medium with unpolished and, subsequently, polished glass Pasteur pipettes. 1 k, 80 k, 150 k, 2500 k cells were seeded in 384-well, 12-well, 6-well plates, 10-cm dishes, respectively. After 12 hrs of seeding, plating media was removed from each well and replaced with complete neurobasal media (neurobasal supplemented with 1% B27, 1% Glutamax, and 0.5% penicillin-streptomycin). Subsequently, approximately 50% of the media in each well was replaced with fresh complete neurobasal media every 3 days. Neurons were maintained at 37° C. under 5% CO₂ until the experimental endpoint.

### Preparation of Adeno-Associated Viral Vectors (AAV)

Crude adeno-associated viral preparation was used. HEK293T cells were obtained from ATCC and cryorecovered in complete Dulbecco's Modified Eagle Medium (DMEM) supplemented with 10% fetal bovine serum (FBS) and 100 U/mL penicillin-streptomycin in 37° C./5% CO₂ incubator (Cat. No. 11965118, 10437028, 15140122, Thermo Fisher). AAV was prepared from HEK293T cells by triple transfection using linear 25 k polyethylenimine (Cat. No. 23966-1, Polysciences, Warrington, PA). 40 μg of total DNA per 150-mm dish (5.7 μg of pAAV, 22.8 μg of pUCmini-iCAP-PHP, and 11.4 μg of pHelper) was used. pUCmini-iCAP-PHP.eB (Addgene #103005, a gift from Dr. Viviana Gradinaru) and pHelper was purchased from Cell Biolabs, Inc. (Cat. No. 340202). Briefly, DNA was diluted in 2 ml OptiMEM (Thermo Fisher, Cat. No. 31985062). 200 μg of PEI prepared was added to the mixture followed by vigorous vortexing. DNA-PEI complex was incubated at RT for 15 min and added drop-wise to cells. On the following day, media was changed to OptiMEM. Five days post transfection, AAV-containing media was filtered, aliquoted and stored in the −80° C. This procedure was used to prepare Camk2a-GFP (pAAV-CW3SL-EGFP, a gift from Dr. Bong-Kiun Kaang, Addgene #61463) to fill neurons for imaging experiments. For NanoLuc experiments, AAV1-AS.mScarlet. NanoLuc was packaged by the Canadian Neurophotonics Platform viral vector vector core facility (RRID:SCR_016477). AAV was added at DIV 3. Unless specified, MOI 5 was used. MOI 1 refers to the volume (in μl) of AAV particles needed=((total number of cells per well)/(number of genome copies (GC)/ml))×1,000.

### Luciferase Assay

Luciferase substrate systems were prepared according to the Manufacturer's instructions. Bright-Glo for Luciferase Assay System (Promega, Cat. No., E2610) was used for any constructs containing firefly luciferase. Nano-Glo Luciferase Assay System (Promega, cat. No., N1110) was used for any constructs containing NanoLuc. The detecting reagent from the manufacturer was diluted 1:1 ratio with OptiMEM or DPBS for Bright-Glo for Luciferase Assay System or the Nano-Glo Luciferase Assay System, respectively. The final volume of detecting reagent mixture was 60 μl and 40 μl for 96-well and 384-well plates, respectively. Cells were lysed directly in the detecting reagent mixture at RT for 5 min with shaking. Plates were read by Biotek Syngery Neo2 plate reader.

### Pharmacology

For initial 384-well plate set up and western blot validation, forskolin (from 25 mM DMSO stock) was used as a positive control and DMSO was used as a vehicle control. For example, at 25 and 2.5 μM forskolin (0.1%, 0.01% DMSO), the corresponding vehicle control was 0.1% and 0.01% DMSO, respectively. Media was changed to NB-27 containing pharmacological agents (concentration specified in figure legends) and processed as described in each section 16 hrs after treatment.

### Immunofluorescence Staining and Confocal Imaging

Coverslips were fixed with 4% paraformaldehyde 4% sucrose in PBS at RT for 15 min and washed three times with PBS. Samples were blocked and permeabilized with 10% bovine serum albumin 0.2% triton-X100 in PBS for 1 hr at RT. Primary antibody incubation (1:1,000) was performed at 4° C. overnight in 5% BSA 0.1% triton-X100 in PBS. Samples were washed three times with 0.1% triton PBS. Secondary antibody incubation (1:500) was performed at RT for 1 hr in 0.1% triton-X100 PBS (containing 50% Intercept blocking PBS buffer, LI-COR, Cat. No., 927-70001). Coverslips were washed three times with 0.1% triton PBS, air dried, and mounted under 10% TBS 90% glycerol mounting media (2.5 μg/ml Hoescht 33342). Coverslips were imaged with an Olympus VS120 slide scanning microscope (Olympus Scientific Solutions Americas, Waltham, MA). Confocal images were acquired with a Leica SP8 confocal microscope (Leica Microsystems) at Northwestern University Biological Imaging Facility.

### Western Blot Analysis

Cell pellets were sonicated in NP40 lysis buffer (1% NP40, 150 mM NaCl, 50 mM Tris, pH 8, 1× Halt). Proteins were separated in 12% Tris-glycine gels and transferred to nitrocellulose membrane (Cat. No. 926-31090, LI-COR, NE, USA). Blots were briefly rinsed with TBS. Total protein was detected using REVERT 700 according to the manufacturer instructions. For detecting individual proteins, unless specified, blots were blocked with 5% milk-containing TBST (0.1% Tween20) for 1 hr at RT. Primary antibodies were added in the same blocking buffer for overnight incubation (1:1,000 rabbit RFP, Rockland, Cat. No., 600-401-379-RTU, and 1:1,000 mouse Psd95 Biolegend Cat. Nol, 810401)). For cFos antibody (1:1,500 mouse anti-cFos, Cat. No., ABE457, Millipore), blots were blocked with 2.5% milk TBST at 135 mM NaCl for 1 hr at RT, instead of 5% milk. Blots were washed three times with TBST for 10 min each at RT. All secondary antibodies were purchased from Li-COR and used at 1:10,000 in TBST for 1 hr at RT. Blots were washed three more times for 10 min each with TBST. Blots were scanned using a LI-COR Odyssey CLx scanner. All quantification was performed using LI-COR Image Studio version 5.2. For densitometry quantification, band signals were normalized to total protein stain in each blot using the equation below.

$$Normalized target signal = \frac{target signal}{lane norm factor} ,$$

$$where lane norm factor = \frac{lane total protein}{{( total protein )}_{blot}}$$

T-test was used to evaluate the treatment effect (1 μM forskoline vs 0.1% DMSO) for RFP, cFos, and Psd95. P-value was corrected by Bonferroni correction. Statistical tests were performed in GraphPad Prism software and p-values were corrected in R using p-adjust function.

### Crude Synaptosome Preparation

Dissected tissues were homogenized using Dounce homogenizers (10 strokes) in 200 μl Syn-PER (Thermo, Cat. No. 87793) supplemented with 1× Halt protease and phosphatase inhibitor cocktail. Lysates were centrifuge at 1200 g for 10 min. S1 supernatant was centrifuged again at 15000 g for 30 min. S2 Supernatant was removed and P2 pellet was washed with 500 μl 0.1M CaCl2 with 1× Halt and centrifuged at 15000 g for 5 min. P2′ pellet was resuspended in 100 μl lysis buffer (1% SDS 125 mM TEAB 75 mM NaCl, 1× Halt) and heated to 85oC for 5 min. 80 μl was used in streptavidin bead enrichment.

### AAV Based Stereotactic Injections

Animals were handled according to protocols approved by the Northwestern University Animal Care and Use Committee. For AAV delivery, P35-40 adult mice were anesthetized using Isoflurane. A midline incision was made using sterile blade to expose the skull. 700 nl of AAV was delivered using an UltraMicroPump (World Precision Instruments, Sarasota, FL) by directing the needle −1.3 mm anterior-posterior, 1.3 mm medial-lateral, and −1.9 mm dorsal-ventral relative to the bregma unilaterally. Following the procedure, the incision was sutured back using nylon thread and the animals were warmed on a heating pad and returned to home cages, with approved post procedure monitoring. Animals were sacrificed and transcardially perfused with 4% PFA 21 days post-surgery for histology. western blots, and proteomics experiments.

### Non-AAV Based Methods of Gene Delivery

### Physical Methods of Gene Delivery:

Biolistic gene delivery: Biolistic delivery can be used as a method to deliver Nanoluc-mScarlet DNA into neurons. In this process, required plasmid is precipitated onto gold microparticles. The ratio of plasmid with the gold particles is optimised for efficient gene delivery. Plasmid: Gold microcarriers are then loaded on a device known as Gene gun (For eg; BioRad PDS-1000/He gene gun) which uses high velocity helium gas to drive Plasmid: Gold microcarriers into the targeted cells. This method is quite challenging for uniform distribution of microcarriers because of high variability between bombarded target cells.
Microneedle system of injecting Nucleic acids: Microneedle based nucleic acid delivery system utilises direct approach of inserting DNA into the target cell. Since naked DNA is prone to degrading enzymes and binding proteins, it cannot be efficiently used without using non-viral vectors. To do this, cultured mature neurons will be used and Nanoluc-mScarlet DNA is conjugated with non-viral nanocarriers such as cationic or ionizable lipid nanoparticles (LNPs), cell penetrating peptides (CPPs, and N-acetylgalactosamine (GalNAc). Conjugated DNA will then be directly injected using very small bore glass needles (outer diameter of usually less than 0.2 μm), for subsequent integration and/or expression. Unlike biolistic gene delivery methods, this approach can ensure precisely controlled delivery into cells.
Electroporation—Another method to introduce Nanoluc-mScarlet can be using electroporation. Electroporation is a flexible technique that uses an electrical field for transient disruption of plasma membranes allowing entry of nucleic acids into the cell. This method can be used in-vivo and in-vitro. The most common mode of electroporation we do in the lab is In-utero electroporation. We have optimised targeting hippocampi using Nanoluc-mScarlet plasmid.

Timed-pregnant E13.5 timed pregnant C57BL6/J females were anaesthetised by using isoflurane in isoflurane induction chamber (3.5% isoflurane and 1.0 LPM oxygen flow). For maintenance isoflurane flow was kept at 2%. The abdomen was incised midline and the uterine horns exposed. The DNA solution (1 μg/μl+0.04% fast green in PBS) was injected into the lateral ventricle of each embryo using a graduated pulled-glass micropipette. The head of each embryo was oriented and placed between tweezer-type electrodes (Nepa 21 Type II super electroporator) and five square electric pulses (100 V, 50 ms pulse length) were passed at 950 ms interval with 10% decay rate. Immediately after electroporation, embryos were rinsed with warm PBS and put back into the abdominal cavity. The wall and skin of the abdominal cavity were sutured and closed, and the embryos were allowed to develop normally till postnatal end points. Animals were perfused at desired postnatal age for expression check.

Lentiviral transduction: Lentiviral vectors are one of the most common non-AAV modes of delivering nucleic acids and are useful to transduce most of the cell types in the central nervous system in-vivo or in-vitro including neurons, astrocytes, adult neural stem cells, oligodendrocytes, and astrocytes. Lentiviruses use reverse transcriptase, that converts viral RNA to dsDNA, and integrase that allows insertion of viral DNA into the host DNA. Insertion process facilitates expression of viral proteins along with proteins encoded by inserted DNA of interest. We have successfully created 1st gen construct lentivirus and tested the expression in primary cortical neurons.

### Chemical Mode of Gene Delivery (Transfection)

These modes of nucleic acid delivery utilize special compounds that interact with the plasma membrane and DNA and allow the vectors enter the cells via endocytosis. There are different classes of compounds/reactions used in chemical transfection.
Calcium phosphate precipitation: It's a classical method of gene delivery which is easy to use and cost-effective. Delivery of DNA is performed by using a combination of buffers which causes co-precipitation of DNA with calcium phosphate. Transfection efficiency is extensively influenced by concentrations of calcium, phosphate and DNA as well as temperature and reaction time.
Lipid based transfection: Liposomal transfection or lipofection is by far the most commonly used gene delivery system. These are synthetic lipid spheres (cationic liposomes) containing polymers linked with fatty acids surrounding an aqueous core which encapsulates small molecules such as DNA of interest. Lipofectamine and turbofectamine (thermofisher) are commonly used for liposomal transfections.

Polymeric carrier based transfection: Cationic polymers are high transfection efficiency polymer-based gene carriers that rely on endocytosis of these synthetic polymers conjugated with DNA of interest. Chitosan, PEI, Polylysine, and poly amino ester commonly used cationic polymers. We use PEI transfection in HEK293T cells to introduce plasmid of interest and generate inhouse AAVs.

### Results

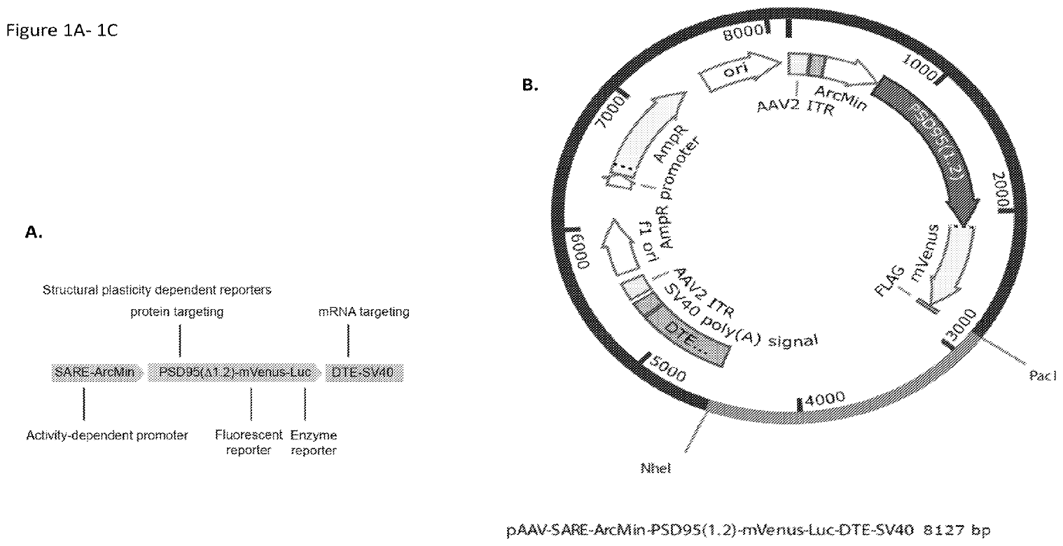

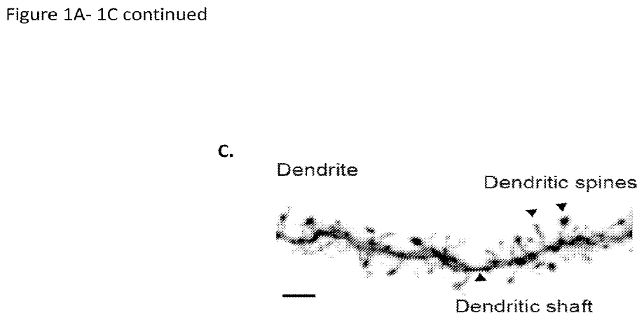

FIG. 1 shows design and assembly of structural plasticity reporters which relies on luciferase reporters under the control of synaptic activity-dependent promoters (Arc in this case) and 3′ untranslated dendrite targeting element (DTE), allowing the amount of luciferase-PSD95 protein expression to be directly correlated with local translation and potentiation of dendritic spines. Because PSD95(Δ1.2) is known to localize to dendritic spine and shaft, the regulation of PSD95-luciferase reporter expression (e.g., translation, localization and degradation) should be similar to the endogenous PSD95. The lack of domain PDZ domains 1-2 ensures that the reporter expression does not interfere with synaptic activity.

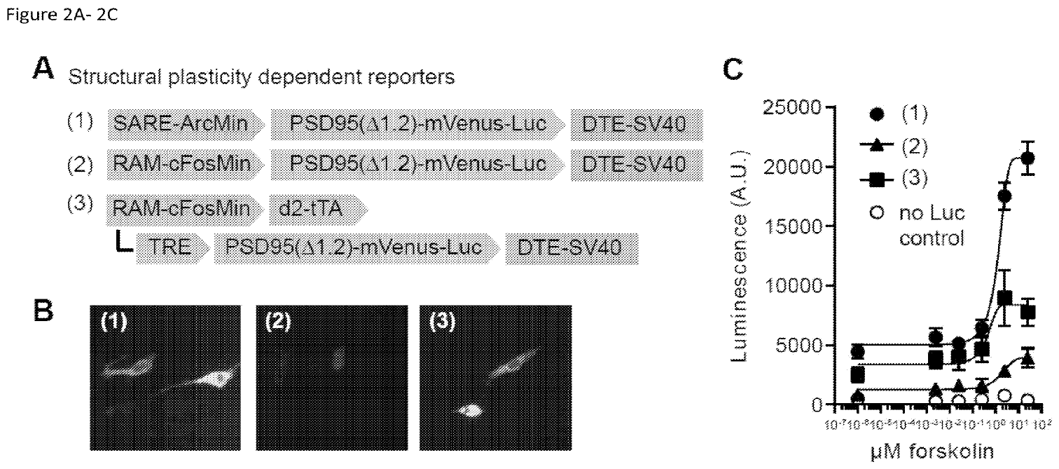

We created three structural plasticity dependent reporters as a proof of concept: Arc, cFos promoters for two major activity-dependent signaling pathways. d2-tTA::TRE reporter amplification cassette. Preliminary characterization of the reporters in HT22 (immortalized mouse hippocampal cell line subcloned from the HT-4 cell line) neuronal cell lines confirm that our luciferase reporters respond to a protein kinase A modulator, forskolin, a known structural plasticity modulator in a dose-dependent manner. The Arc pathway (FIG. 2. construct (1)) shows the strongest response to FSK stimulation compared to the cFos pathway (construct (2)). Construct (3), which is another version of construct (2) in an amplification cassette, shows a similar dose-response behavior to construct (2) but with greater signal. Results from these pilot experiments indicated that the construct (1) is most sensitive and we selected it for follow up assays.

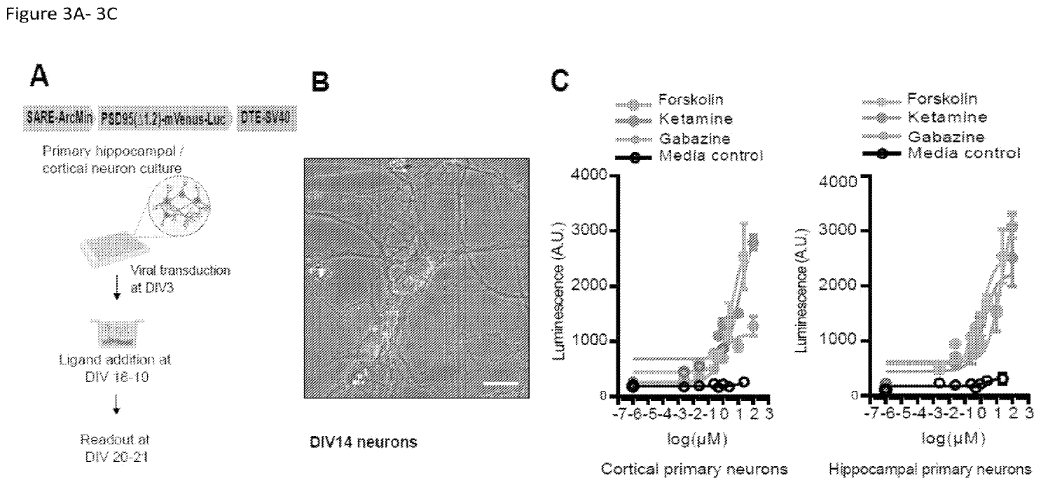

Since cell line cannot capture the complexity of neural plasticity, we validated our assay for high throughput screening in primary neuronal cultures. We used known plasticity modulators (Forskolin, Ketamine, and Gabazine) and a negative control of media with no pharmacological agent to test construct (1) in a 96-well plate format (FIG. 3). The aim of this experiment was to observe differential luciferase activity in response to different pharmacological compounds in mature primary neurons. Because each cell type in the brain has a unique circuitry, we tested two different types of primary cultures-hippocampal and cortical neurons. We observed that the dose-response is cell-type specific. Preliminary evaluation of the assay in primary culture provided us useful information about technical variability, dynamic range, and cutoff analysis.

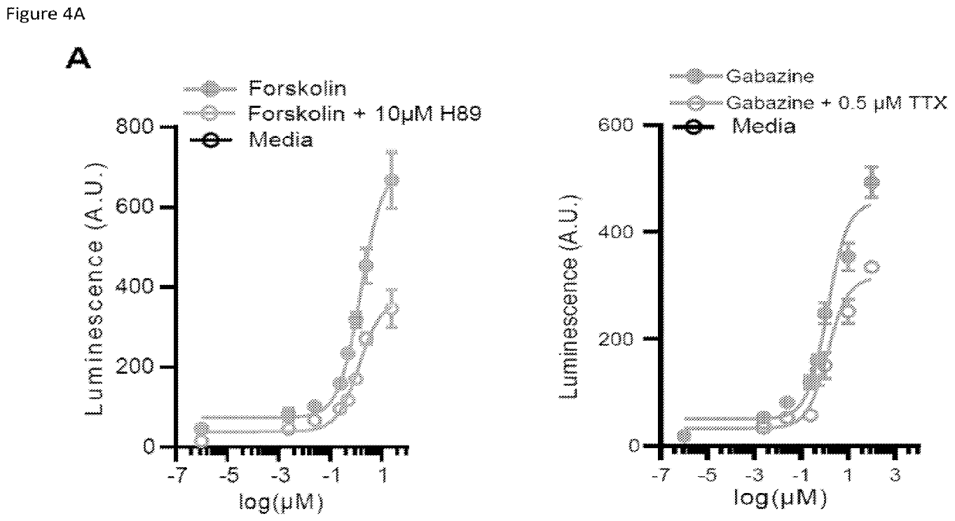

To confirm dependence of luminescence increase on the activity of known modulator, we used their corresponding pathway specific inhibitors (e.g., Forskolin acts via Protein Kinase A, therefore we used a PKA inhibitor, H89). We found that the luciferase activity in response to a known modulator is reduced upon application of their corresponding inhibitors in primary cortical neurons (FIG. 4). The results highlight the flexibility of this activity-sensor to identify both plasticity promoting and dampening modulators in a pathway specific manner.

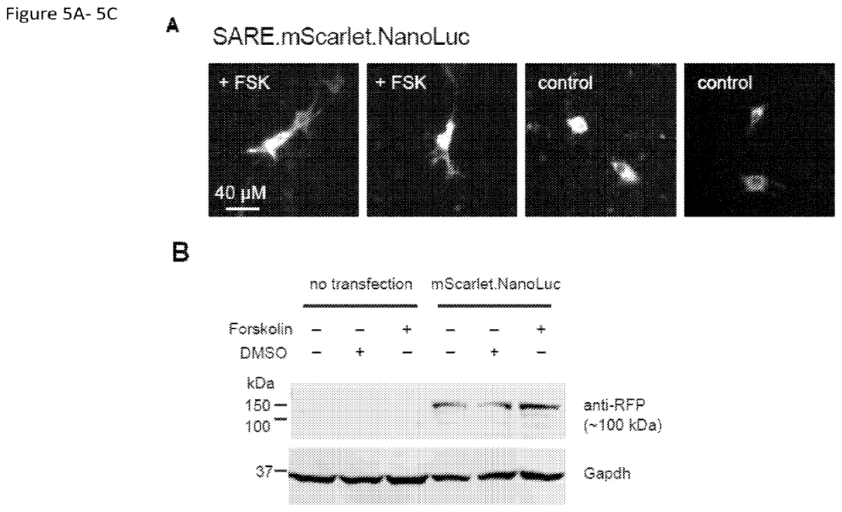

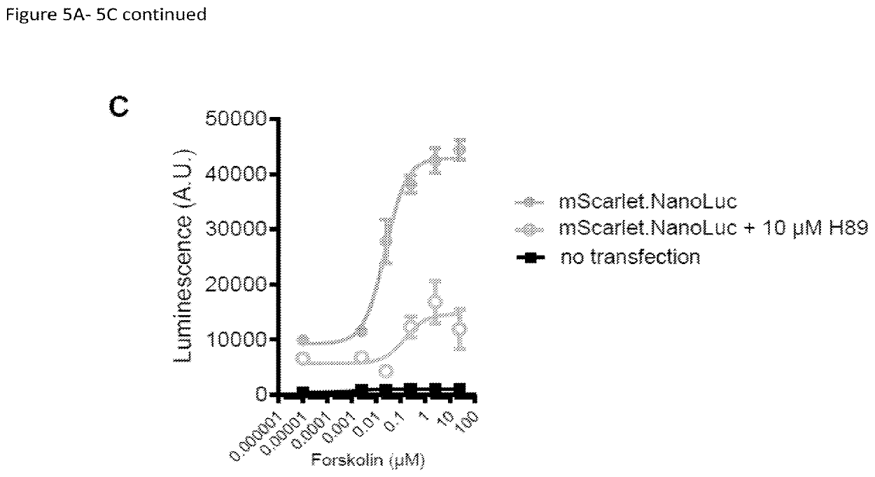

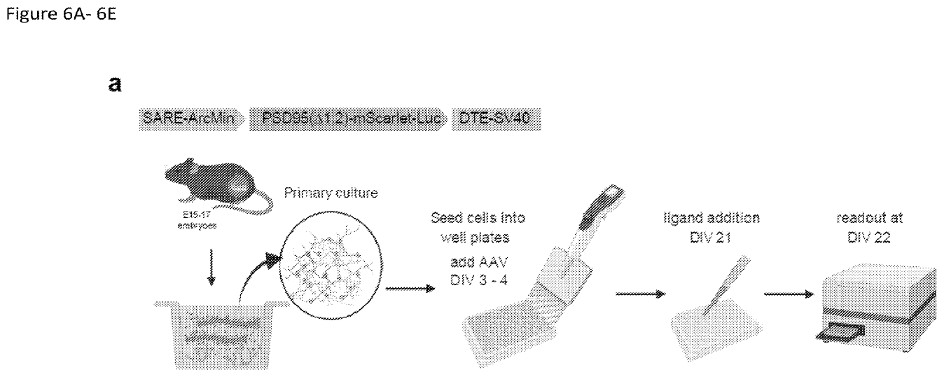

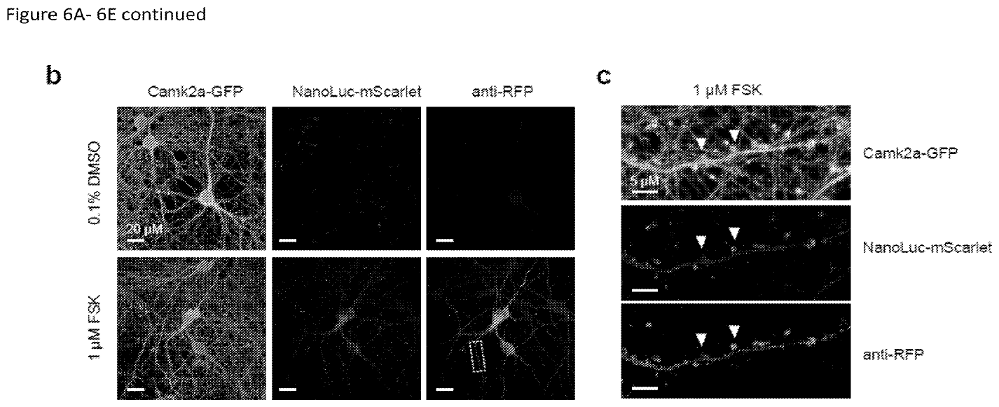

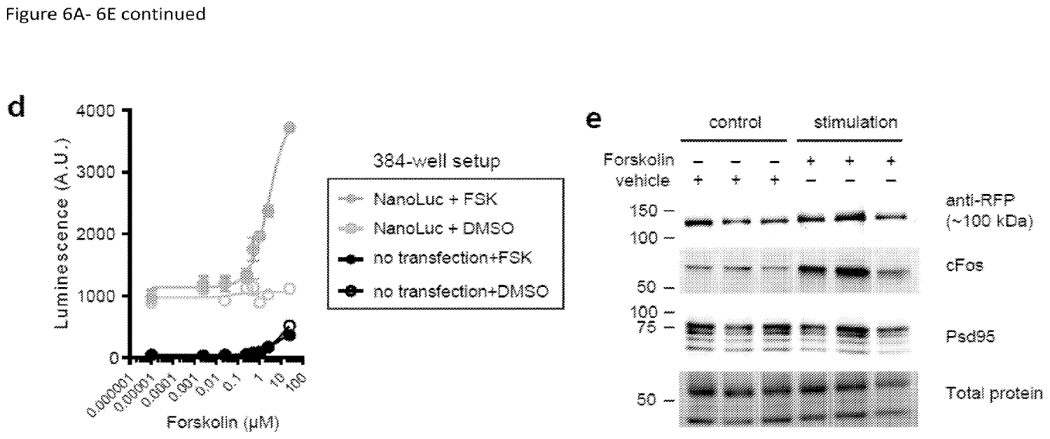

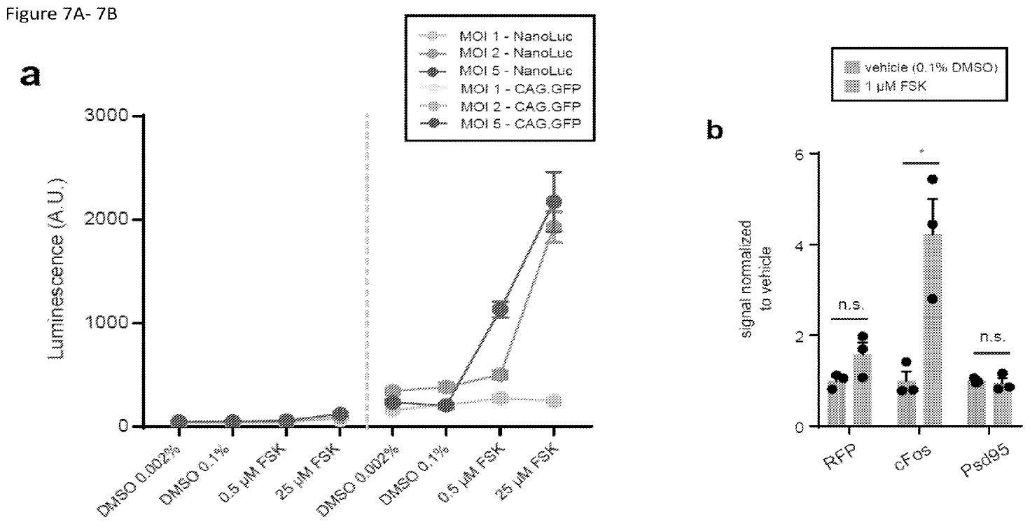

We next modified SARE-Arcmin-Luciferase construct and created a second generation construct with enhanced sensitivity, readability, ability to get packaged easily in to an AAV, and visualized better for imaging. We modified the Arc-based construct (1) by swapping the firefly luciferase for Nanoluciferase and switched from mVenus to mScarlet fluorophore reporter. We tested the second generation construct in HT22 cells using imaging and Western blot. The second generation sensor showed enhanced readout in luminescence (FIG. 5).

We further tested the 2^{nd} gen construct (designated as Nanoluc-mScarlet from now on) in mature cortical neuron cultures. These improvements reduced construct size, enhanced its activity and facilitated imaging with a bright red fluorophore. NanoLuc and its substrate provided extremely sensitive and stable signal in a 384-well plate system, where the number of cells is limited. This set of experiments showed robust dose-dependent response of the modified construct to PKA activator forskolin (FSK) and confirm ease of imaging. In line with the functional validation of the screen, we observed higher expression of mScarlet reporter in cells and increased protein expression.

Based on the previous pilot tests, we confirmed that the 2^{nd} gen construct is superior than the 1^{st} gen construct and can be used for high-throughput screening in a 384-well plate format. Therefore, we packaged this construct in an adeno-associated virus (AAV) commercially from Neurophotonics (1.5×10¹³ Genome copy). To validate the potency and efficacy of packaged Nanoluc-mScarlet AAV, we titrated it in primary neurons. The transduction efficiency of an AAV is determined by MOI (multiplicity of infection) which determine the number of viral particles required for a given number of cells to elicit optimal response/infection. We observed that MOI 5 of the packaged AAV is enough for a robust luminescence response without any toxicity. We used CAG-GFP AAV as a viral control to optimize background as well as toxicity.

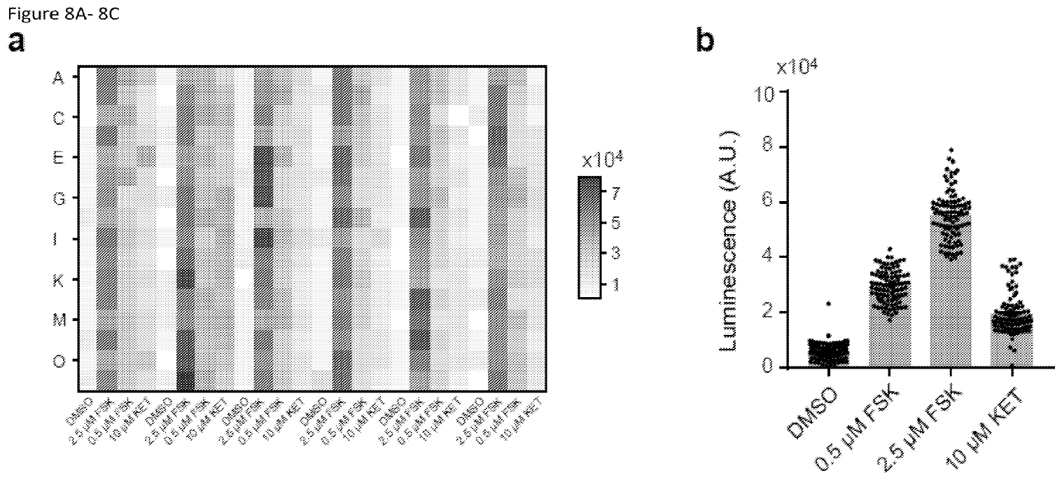

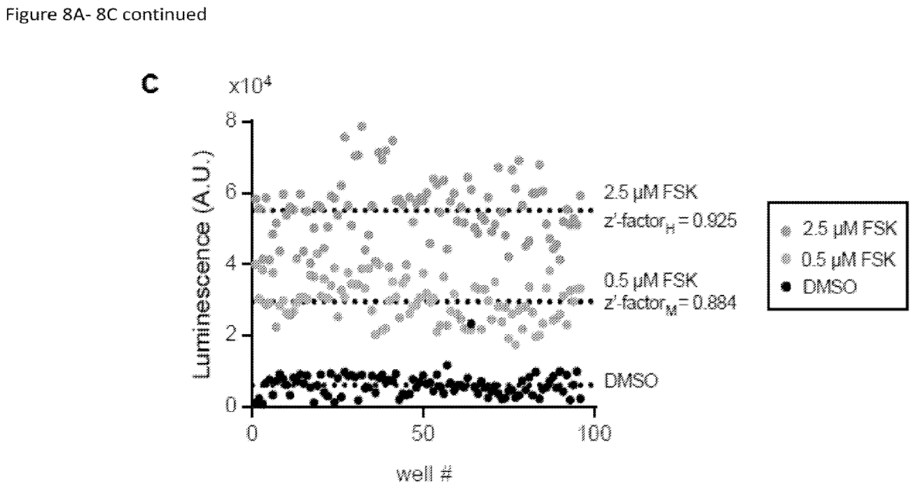

Next, we conducted a preliminary assessment to adapt this construct for high-throughput screen, by following the NIH Assay Guidance Manual. The interleaved plate layout was used to detect any systematic signal uniformity issues in data across wells (e.g., edge and drift effects from evaporation, detecting reagent stability, respectively). In this experiment, we used 0.1% DMSO as min, 0.5, and 2.5 μM FSK as mid and max. Drift and edge effects were not significant across the plate, because we did not observe higher signal along wells on the edges or from left to right wells. Z′-factor was calculated to evaluate signal separate and reproducibility. Robust z′-factors of both mid and high doses of FSK response compared to DMSO indicate sufficient separation between positive and negative controls indicated efficacy of the screening method to differentiate background vs signal (FIG. 8).

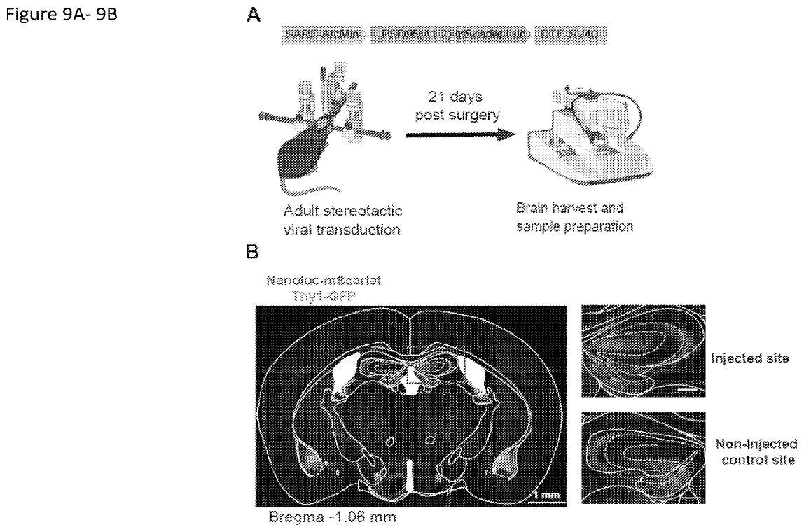

We also validated the expression of Nanoluc-mScarlet in mice brain to further optimize the protocol for imaging plasticity-dependent NanoLuc in mice. We bilaterally injected AAV encoding the NanoLuc construct unilaterally in hippocampus (−1.3 mm anterior-posterior, 1.3 mm medial-lateral, and −1.9 mm dorsal-ventral relative to the bregma) of adult mice. The objective of this experiment was to visualize Nanoluc-mScarlet without immunoenhancement for future synapse and dendritic spine genesis experiments, or other applications (FIG. 9). The results from this set of experiments opened the path towards using the construct for monitoring in vivo neural response to neuroplasticity enhancers in living animals non-invasively, for example using IVIS Spectrum imaging technology.

### Example II—Activated Synaptic Proteomic Screen

We also extended the application of our neuronal potentiation-sensing construct to specifically isolate and target the total postsynaptic proteome in activated synapses. Enzyme-catalyzed proximity labeling is an emerging new tool to study spatial and protein-protein interactions in cells. A promiscuous labeling enzyme is used as a targeting aid by genetic fusion with specific proteins or subcellular compartment targeting tags. Covalent tagging of endogenous proteins within a few nanometers of the enzyme is initiated by the addition of specific substrate, such as biotin.

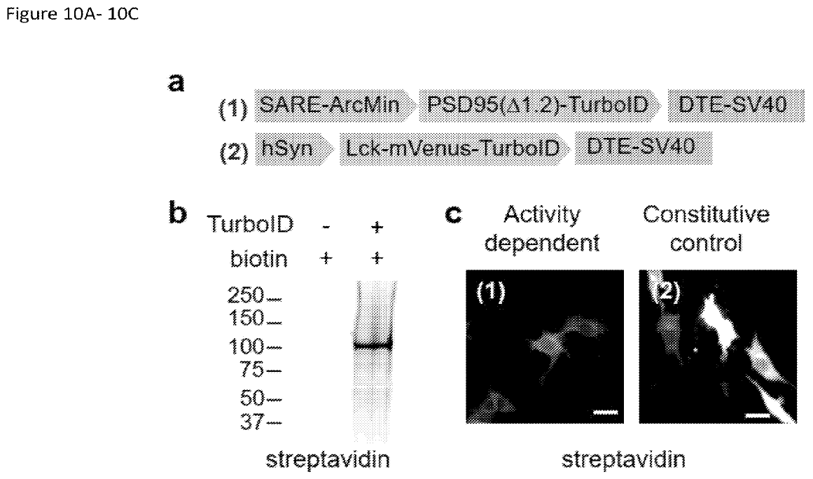

To do this, we modified the 1^{st} generation construct by replacing luciferase sequence with TurboID, an engineered biotin ligase. This modification allows us to specifically target and label proteins in dendritic spines upon activation and broaden the utility of our construct, by swapping out the effector gene. We also generated an independent constitutive control-membrane targeting control (membrane anchor LCK sequence, which we have extensive experience with via imaging, proteomics, and electron microscopy). We first validated biotinylation of proteins mediated by these constructs in HT22 cells (FIG. 10).

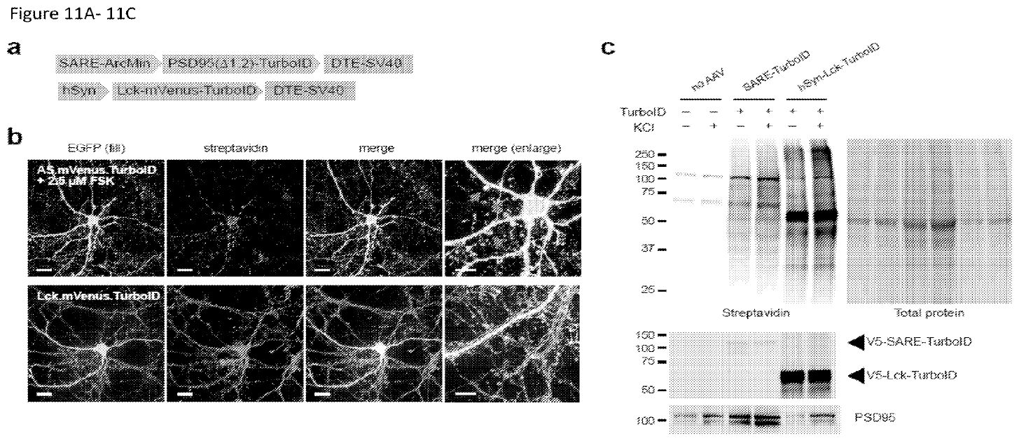

Next, we determined proximity labelling of proteins by our construct based on two parameters-ability to specifically label synaptic proteins and snapshotting the subtle/robust changes in total synaptic proteome turnover after potentiation. We validated these parameters using two different tools: visualization of biotinylated proteins in cells and change in expression of totally biotinylated proteins upon neuronal activity induction (FIG. 11).

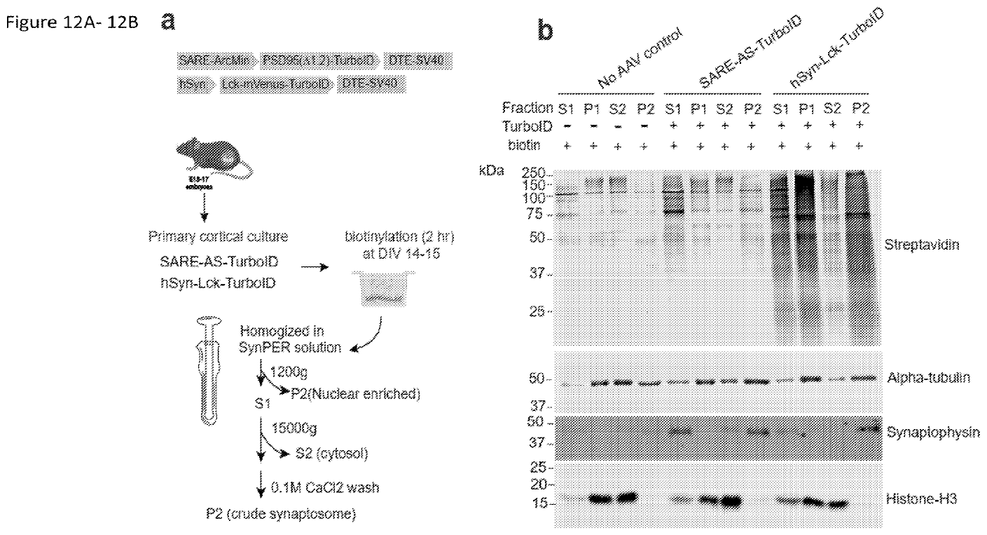

We furthermore, added another layer of validation of compartment specificity of our constructs by performing subcellular fractionation. With the existing working pipeline of fractionation in our lab, we show that the biotinylated proteins in the cells transduced by SARE-Arc-TurboID are highly enriched in synaptosomal fraction in comparison with cytosolic or nuclear fraction (FIG. 12).

### REFERENCES

1. Maiti, P., Manna, J., Ilavazhagan, G., Rossignol, J. & Dunbar, G. L. Molecular regulation of dendritic spine dynamics and their potential impact on synaptic plasticity and neurological diseases. Neuroscience & Biobehavioral Reviews 59, 208-237 (2015).
2. Mahalakshmi, A. M., Ray, B., Tuladhar, S., Hediyal, T. A., Raj, P., Rathipriya, A. G., Qoronfleh, M. W., Essa, M. M. & Chidambaram, S. B. Impact of Pharmacological and Non-Pharmacological Modulators on Dendritic Spines Structure and Functions in Brain. Cells 10, 3405 (2021).
3. Nishiyama, J. Plasticity of dendritic spines: Molecular function and dysfunction in neurodevelopmental disorders. Psychiatry and Clinical Neurosciences 73, 541-550 (2019).
4. Berry, K. P. & Nedivi, E. Spine Dynamics: Are They All the Same? Neuron 96, 43-55 (2017).
5. Zuo, Y., Lin, A., Chang, P. & Gan, W.-B. Development of Long-Term Dendritic Spine Stability in Diverse Regions of Cerebral Cortex. Neuron 46, 181-189 (2005).
6. Penzes, P., Cahill, M. E., Jones, K. A., VanLeeuwen, J.-E. & Woolfrey, K. M. Dendritic spine pathology in neuropsychiatric disorders. Nature Neuroscience 14, 285-293 (2011).
7. Herms, J. & Dorostkar, M. M. Dendritic Spine Pathology in Neurodegenerative Diseases. Annual Review of Pathology: Mechanisms of Disease 11, 221-250 (2016).
8. Barón-Mendoza, I., Maqueda-Martinez, E., Martinez-Marcial, M., De la Fuente-Granada, M., Gómez-Chavarin, M. & González-Arenas, A. Changes in the Number and Morphology of Dendritic Spines in the Hippocampus and Prefrontal Cortex of the C58/J Mouse Model of Autism. Frontiers in Cellular Neuroscience 15, 726501 (2021).
9. Dorostkar, M. M., Zou, C., Blazquez-Llorca, L. & Herms, J. Analyzing dendritic spine pathology in Alzheimer's disease: problems and opportunities. Acta Neuropathologica 130, 1-19 (2015).
10. Wu, M., Minkowicz, S., Dumrongprechachan, V., Hamilton, P. & Kozorovitskiy, Y. Ketamine Rapidly Enhances Glutamate-Evoked Dendritic Spinogenesis in Medial Prefrontal Cortex Through Dopaminergic Mechanisms. Biol Psychiatry 89, 1096-1105 (2021).
11. Lukasiewicz, K., Baker, J. J., Zuo, Y. & Lu, J. Serotonergic Psychedelics in Neural Plasticity. Frontiers in Molecular Neuroscience 14, (2021).
12. Shao, L.-X., Liao, C., Gregg, I., Davoudian, P. A., Savalia, N. K., Delagarza, K. & Kwan, A. C. Psilocybin induces rapid and persistent growth of dendritic spines in frontal cortex in vivo. Neuron 109, 2535-2544.e4 (2021).
13. Rose, S. P. R. ‘Smart Drugs’: do they work? Are they ethical? Will they be legal? Nature Reviews Neuroscience 3, 975-979 (2002).

1. An activity-dependent regulated polynucleotide capable of detecting dendritic potentiation comprising one or more of the following:
(a) an activity-response element,
(b) an activity-dependent promoter,
(c) a subcellular targeting motif,
(d) a fluorescent reporter or biotinylating reporter,
(e) an enzyme reporter, and
(f) an mRNA targeting element.

2. The polynucleotide of claim 1, wherein the polynucleotide comprises at least two or more of (a)-(f).

3. The polynucleotide of claim 1 or 2, wherein the polynucleotide comprises three or more of (a)-(f).

4. The polynucleotide of any one of the preceding claims, wherein the polynucleotide comprises (a)-(f).

5. The polynucleotide of any one of the preceding claims, wherein the activity-response element comprises a synaptic activity response element of (SARE) or a robust activity marking system (pRAM).

6. The polynucleotide of claim 5, wherein the activity response element is SARE, and SARE comprises SEQ ID NO:1 or a sequence having 90% similarity to SEQ ID NO:1.

7. The polynucleotide of claim 5, wherein the activity response element is pRAM, and pRAM comprises SEQ ID NO:2.

8. The polynucleotide of any one of the preceding claims, wherein the activity dependent promoter is a synaptic activity-dependent promoter selected from the group consisting of Arc, cFos, Npas4, Egr1, and cJun.

9. The polynucleotide of claim 8, wherein the synaptic activity-dependent promoter is Arc, preferably comprising SEQ ID NO:3 or a sequence having 90% similarity to SEQ ID NO:3.

10. The polynucleotide of any one of the preceding claims, wherein the subcellular targeting motif is a synaptically localized tag comprising Psd95(Δ1.2), optionally wherein the synaptically localized tag comprises SEQ ID NO: 5 or a sequence having at least 90% similarity to SEQ ID NO:5.

11. The polynucleotide of any one of the preceding claims wherein the fluorescent reporter is a fluorescent acceptor, and the enzyme reporter is a bioluminescent donor.

12. The polynucleotide of claim 11 wherein the fluorescent reporter is selected from the group consisting of mVenus and mScarlet.

13. The polynucleotide of claim 12, wherein the fluorescent reporter comprises SEQ ID NO:7-10 or a sequence having 90% similarity to any one of SEQ ID NO:7-10.

14. The polynucleotide any one of the preceding claims, wherein the polynucleotide comprises an enzyme reporter.

15. The polynucleotide of claim 14, wherein the enzyme reporter comprises is selected from firefly luciferase and Nanoluciferase.

16. The polynucleotide of claim 15, wherein the enzyme reporter comprises any one of SEQ ID NO:11-14 or a sequence having at least 90% sequence identity to SEQ ID NO:11-14.

17. The polynucleotide of any one of the preceding claims, wherein the subcellular targeting motif is a mRNA targeting motif in the 3′ untranslated region (UTR).

18. The polynucleotide of claim 17, wherein mRNA targeting motif comprises SEQ ID NO: 17-18 or a sequence having at least 90% sequence identity to SEQ ID NO: 17-18.

19. The polynucleotide any one of the preceding claims, wherein the mRNA targeting motif is a dendritic targeting element (DTE), optionally wherein the DTE comprises SEQ ID NO: 17 or a sequence having 90% identity to SEQ ID NO:17.

20. The polynucleotide of any one of the preceding claims, wherein the enzyme reporter is replaced by a biotin ligase.

21. The polynucleotide of claim 20, wherein the biotin ligase is SEQ ID NO: 15-16 or a sequence having at least 90% sequence similarity to SEQ ID NO:15-16.

22. The polynucleotide of any one of the preceding claims, further comprising an optional reporter amplification cassette.

23. A construct capable of introducing the polynucleotide into a host cell, the construct comprising the polynucleotide of any one of claims 1-22.

24. The construct of claim 23, wherein the construct is a vector.

25. A virus vector comprising the polynucleotide of any one of claims 1-23 or the construct of claims 12-13.

26. The virus of claim 25, wherein the virus is an adeno-associated virus or lentivirus.

27. A host cell expressing the polynucleotide of any one of claims 1-23, the construct of claim 24.

28. The host cell of claim 27, wherein the host cell is a neuron or a neuron-like cell.

29. A high throughput method of testing a therapeutic molecule capable of altering dendritic potentiation, the method comprising:
(a) contacting a cell expressing the polynucleotide of any one of claims 1-22 or the construct of claim 23 or 24 with a therapeutic molecule; and
(b) detecting the reporter expression in the cell, wherein the expression of the reporter is indicative of dendritic or synaptic potentiation.

30. The high throughput method of claim 29, wherein the cell is a neuron or a neuron-like cell.

31. The method of claim 20 or 30, where the reporter is a fluorescent, enzymatic or biotinylating reporter, and the method comprises:
detecting fluorescence by immunofluorescence;
detecting luminescence by high-throughput means;
detecting biotinylation by mass spectrometry based proteomics.

32. The method of any one of claims 18-20, wherein the detecting of a reporter signal indicates that the therapeutic molecule is a neuroactive agent that modulates structural plasticity in the dendritic spines of neurons.

33. The method of any one of claims 29-32, wherein the method comprises:
contacting in separate containers multiple therapeutic agents at different concentrations with different wells comprising the cells of (a) in a multiwell plate.

34. The method of claim 33, wherein the method further comprises,
detecting a signal from each well of a multiwell plate wherein reporter activity is indicative of dendritic or synaptic potentiation.

35. The method of claim 34, wherein the multiwell plate is a 48-well plate, a 96-well plate, or a 384 well plate.

36. A kit comprising as components:
(a) the polynucleotide of any one of claims 1-22 or the construct of claim 23 or 24; and
(b) instructions for use in detecting the reporter expression in a cell, wherein the expression of the reporter is indicative of dendritic or synaptic potentiation of the cell.

37. The kit of claim 36, wherein the kit further comprises a multiwell plate.
